SEQUENCE LISTING (3 sequences)

SEQ ID NO: 1 - DNA, 40 bp, Listeria monocytogenes
acaaatgtgccgccaagaaaaggttacaaagatggaaatg

SEQ ID NO: 2 - DNA, 36 bp, Listeria monocytogenes
aaatgtgccgccaagaaaaggttacaaagatggaaa

SEQ ID NO: 3 - DNA, 95 bp, Artificial
  Synthetic DNA
tttcttnncnncacattcattcacattcacattcacattcacattcacattcacattcacattcacattcacattcatttccatctttntaacct

(19) United States
(12) Patent Application Publication
(10) Pub. No.: US 2010/0184159 A1
(43) Pub. Date: Jul. 22, 2010

(54) Compositions, Methods, and Kits for Selective Amplification of Nucleic Acids

(75) Inventors: Vissarion AIVAZACHVILI, Oakland, CA (US); Konard Faulstich, Salem-Neufrach (DE); Tony Tran, Mountain House, CA (US)

Correspondence Address: LIFE TECHNOLOGIES CORPORATION;C/O INTELLEVATE, P.O. BOX 52050, MINNEAPOLIS, MN, 55402, US

(73) Assignee: APPLIED BIOSYSTEMS, LLC, Carlsbad, CA (US)

(21) Appl. No.: 12/692,557

(22) Filed: Jan. 22, 2010

Related U.S. Application Data
(62) Division of application No. 11/331,599, filed on Jan. 12, 2006, now abandoned.
(60) Provisional application No. 60/643,763, filed on Jan. 12, 2005.

(51) Int. Cl. C12P 19/34

(52) U.S. Cl. 435/91.5

(57) ABSTRACT

The current teachings are directed to compositions, methods, and kits for selectively amplifying and for detecting target sequences. In some embodiments, a circularizable probe and/or a probe pair are disclosed for selectively amplifying target sequences. Methods for selectively amplifying target sequences are also disclosed, as are methods for detecting selectively amplified target sequences. Certain embodiments of the disclosed methods comprise a circularizable probe, a probe pair, comprising a first probe and a second probe, or both. In certain embodiments, a multiplicity of different circularizable probes, a multiplicity of different probe sets, or a multiplicity of different circularizable probes and a multiplicity of different probe sets are provided to selectively amplify or to detect a multiplicity of different target sequences, typically in a multiplex reaction. According to certain disclosed methods, surrogates of the target sequences are selectively amplified, including without limitation ligated probes, first amplification products, second amplification products, or combinations thereof. In some embodiments, selectively amplified target sequences or their surrogates are detected, directly or indirectly, indicating the presence of the corresponding target sequence. Kits to facilitate the performance of the disclosed methods are also provided.

### CROSS-REFERENCE TO RELATED APPLICATIONS

This application claims a priority benefit under 35 U.S.C. §119(e) from U.S. Patent Application No. 60/643,763, filed Jan. 12, 2005 and U.S. patent application Ser. No. 11/331,599, filed Jan. 12, 2006, which are incorporated herein by reference.

### FIELD

The present teachings generally relate to nucleic acid amplification and detection. More specifically, the disclosed compositions, methods, and kits are useful in selectively amplifying and/or detecting specific nucleic acid targets in the presence of non-target nucleic acids using a probe or a probe pair that typically comprise three of the four nucleotides, but not all four.

### BACKGROUND

Many current nucleic acid amplification methods, such as the polymerase chain reaction (PCR) can suffer sensitivity and specificity limitations due to the presence of non-target nucleic acids that may be present in the sample. In some instances, these “background” sequences may be present in vast excess compared to the nucleic acid sequences of interest. Reagents and methods for selectively amplifying and detecting target sequences in the presence of high background would be useful. For example but not limited to, detecting pathogens or indicator organisms in food, environmental samples, including without limitation, soil, air, and water samples, or samples suspected to contain a bioterrorism agent (see, e.g., Center for Disease Control list at bt.cdc.gov/Agent/Agentlist on the world wide web); forensics samples that potentially contain nucleic acid from more than one source; and clinical samples, such as blood, serum, sputum, tumor or other biopsy material, and the like, that potentially contain relatively small amounts of target sequences.

### SUMMARY

The present teachings are directed to compositions, methods, and kits for selectively amplifying and for detecting target sequences, typically in the presence of “background”, e.g., non-target nucleic acids. In certain embodiments, the target sequence(s) represents a minority species or an extreme minority species in a particular nucleic acid population, such as a sample. According to the current teachings, such targets can be selectively amplified, typically without the need for extensive prior removal of background.

In some embodiments of the current teachings, target-specific probes are provided. Certain probe embodiments comprise: (a) a first target-complementary portion, (b) a second target-complementary portion, and (c) a spacer portion; wherein the first target-complementary portion comprises three of the four nucleotide bases: (1) A, (2) C, (3) G, and (4) T, U, or T and U, but not the fourth nucleotide base. The three nucleotide bases that are present in the first target-complementary portion are also typically present in the second target-complementary portion and the spacer portion, but the fourth nucleotide base that is absent from the first target-complementary portion is not present. In some embodiments, a target-complementary portion of the disclosed probes further comprises a universal base. In other embodiments, probe pairs are provided wherein each probe of the probe pair comprises a target-complementary portion that comprises, consists of, or consists essentially of three of the four nucleotide bases: (1) A, (2) C, (3) G, and (4) T, U, or T and U, but not the fourth nucleotide base.

According to some embodiments, methods for detecting target nucleic acids are provided that employ a target-specific probe, a target-specific probe pair, or both. In other embodiments, methods for selectively amplifying target sequences are provided comprising a target-specific probe, a target-specific probe pair, or both. Certain embodiments comprise a multiplicity of different target-specific probes, a multiplicity of different probe pairs, or both, for selectively amplifying or for detecting a multiplicity of different target sequences.

Certain of the disclosed methods comprise hybridizing a probe comprising at least one target-complementary portion with a target sequence. In some embodiments, the target-complementary portion of the probe comprises three of the four nucleotide bases, but not the fourth nucleotide base; while in other embodiments, the target-complementary portion further comprises a universal base, either in place of the fourth nucleotide base, for increasing probe specificity, or both. Such methods also comprise a step for selectively amplifying the hybridized probe or a surrogate of the hybridized probe, including without limitation, a first amplification product; and a step for detecting the amplified probe or its surrogate. Some methods further comprise: a step for denaturing a double-stranded target sequence or a double-stranded nucleic acid comprising the target sequence; a step for generating a strand invasion structure; a step for ligating (i) a probe comprising a first- and a second target-complementary portion, (ii) a probe pair, comprising a first probe and a second probe that each comprise a target-complementary portion, (iii) a gap oligonucleotide and either the probe or the probe pair, of (iv) combinations thereof; a step for releasing the ligated probe; a step for amplifying a first amplification product, a second amplification product, surrogates of either, or combinations thereof; a step for generating luminescence; a step for detecting; or combinations thereof.

Also disclosed are methods for selectively amplifying target sequences or for detecting target sequences, typically in the presence of a large excess of non-target sequences. Such methods employ probes or probe pairs of the current teachings that: (a)(i) comprise, (ii) consist of, or (iii) consist essentially of: (b) three of the four nucleotide bases, but not the fourth nucleotide base; and (c) can, but need not include a universal base; and also a nucleotide-deficient first reaction composition.

According to the selective amplification methods and the detection methods of the present teachings, a probe(s) is hybridized with the target sequence and then selective amplification occurs in a nucleotide-deficient first reaction composition. Typically, the hybridized probe or a hybridized probe pair is ligated to form a ligated probe prior to the selective amplification, but not always. The first reaction composition is nucleotide-deficient in that it contains three of the four nucleotides, but not the fourth. Those in the art will appreciate that the absence of the fourth nucleotide base in the first reaction composition will inhibit the amplification of those sequences that contain all four nucleotides. Hence, those sequences that contain nucleotide bases that are the complement of the nucleotide triphosphates present in the nucleotide-deficient reaction composition, but not the missing nucleotide, are selectively amplified while other sequences comprising all four nucleotides are not amplified.

In some of the disclosed methods, the target sequence is from a microorganism. In some embodiments, the target sequence is present in an environmental sample, including without limitation, a soil sample, an air sample, or a water sample, or a surface swab. In other embodiments, the target sequence is from a mammal, such as a human, and the sequence is typically an indicator of a particular condition or physiological state in that mammal, for example but not limited to, cancer or a genetic disorder. In certain circumstances, a human target sequence is selectively amplified and/or detected for forensics evaluation or human identification.

Kits for performing certain of the instant methods are also disclosed. Certain kit embodiments include probes comprising three of the four nucleotide bases: (1) A, (2) C, (3) G, and (4) T, U, or T and U, but not the fourth nucleotide base; such probes can, but need not, further comprise a universal base. Certain kit embodiments include a probe and/or a probe pair consisting of, or consisting essentially of, three of the four nucleotide bases: (1) A, (2) C, (3) G, and (4) T, U, or T and U, but not the fourth nucleotide base. Certain kit embodiments include a probe and/or a probe pair consisting of, or consisting essentially of, a universal base and three of the four nucleotide bases: (1) A, (2) C, (3) G, and (4) T, U, or T and U, but not the fourth.

These and other aspects of the present teachings are set forth herein.

### BRIEF DESCRIPTION OF THE DRAWINGS

FIG. 1: depicts an exemplary probe of the present teachings. The probe 5 is phosphorylated at its 5′-end (shown as “p”) and has a free hydroxyl group at its 3′-end (shown as “OH”). “N” represents a universal base, for example but not limited to 5-nitroindole. The line shown in the middle of the target sequence 5 is for illustration purposes only and does not signify a space between the two segments of the target sequence.

FIG. 2: depicts an exemplary probe pair of the current teachings. The line shown in the middle of the target sequence 5 is for illustration purposes only and does not signify a space between the two segments of the target sequence.

FIG. 3: schematically depicts an illustrative embodiment of the current teachings.

FIGS. 4A and 4B: schematically depict an illustrative embodiment of the current teachings.

FIGS. 5A-D: schematically depicts an illustrative embodiment of the current teachings. The curved arrow symbol and “PPi” indicate the release of inorganic pyrophosphate by the corresponding reaction.

FIG. 6: schematically depicts an illustrative embodiment of the current teachings. “FTCP”: first target-complementary portion; “STCP”: second target-complementary portion; “1AP”: first amplification product; “PPi”: inorganic pyrophosphate; “APS” represents adenosine 5′-phosphosulfate.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. For example, “a probe” means that more than one probe can be present; for example, one or more copies of a particular probe species, as well as one or more versions of a particular probe type. Also, the use of “comprise”, “comprises”, “comprising”, “contain”, “containing”, “contains”, “include”, “includes”, and “including” are not intended to be limiting. The term and/or is intended to have its conventional meaning, i.e., that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. In the event that one or more of the incorporated literature and similar materials differs from or contradicts this application, including but not limited to defined terms, term usage, described techniques, or the like, this application controls.

### I. DEFINITIONS

The term “affinity tag” as used herein refers to a component of a multi-component complex, wherein the components of the multi-component complex specifically interact with or bind to each other. Exemplary multiple-component affinity tag complexes include without limitation, ligands and their receptors, for example but not limited to, avidin-biotin, streptavidin-biotin, and derivatives of biotin, streptavidin or avidin, including without limitation, 2-iminobiotin, desthiobiotin, NeutrAvidin (Molecular Probes, Eugene, Oreg.), CaptAvidin (Molecular Probes), and the like; binding proteins/peptides and their binding partners, including without limitation, maltose-maltose binding protein (MBP), calcium-calcium binding protein/peptide (CBP); epitope tags, for example but not limited to c-MYC (e.g., EQKLISEEDL), HA (e.g., YPYDVPDYA), VSV-G (e.g., YTDIEMNRLGK), HSV (e.g., QPELAPEDPED), V5 (e.g., GKPIPNPLLGLDST), and FLAG Tag™ (e.g., DYKDDDDKG), and their corresponding anti-epitope antibodies; haptens, for example but not limited to dinitrophenol (“DNP”) and digoxigenin (“DIG”), and their corresponding antibodies; aptamers and their binding partners; poly-His tags (e.g., penta-His and hexa-His) and their binding partners, including without limitation, corresponding metal ion affinity chromatography (IMAC) materials and anti-poly-His antibodies; fluorophores and their corresponding anti-fluorophore antibodies; and the like. In certain embodiments, affinity tags are part of a separating means, part of a detecting means, or both.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The term “corresponding” as used herein refers to a specific relationship between the elements to which the term refers. For example, a probe of the current teachings corresponds to the target sequence with which it specifically hybridizes, and vice versa. A first probe of a probe pair corresponds with the second probe of that probe pair. A primer is designed to anneal with the primer-binding portion of a corresponding ligated probe, a corresponding first amplification product, a corresponding second amplification product, or combinations thereof. The target-complementary portions of the instant probes or probe sets are designed to hybridize with a complementary or substantially complementary region of the corresponding target sequence or the complement of the target sequence. A particular affinity tag binds to the corresponding affinity tag, for example but not limited to, biotin binding to streptavidin. A particular hybridization tag anneals with its corresponding hybridization tag complement; and so forth.

The term “enzymatically active mutants or variants thereof” when used in reference to an enzyme or enzyme type, such as a polymerase, a ligase, a nuclease, an extending enzyme, an amplification means, or the like, refers to one or more polypeptide derived from a corresponding enzyme or enzyme type that retains at least some of the desired enzymatic activity, such as ligating, amplifying, or digesting, as appropriate. Also within the scope of this term are: enzymatically active fragments, including but not limited to, cleavage products, for example but not limited to Klenow fragment, Stoffel fragment, or recombinantly expressed fragments and/or polypeptides that are smaller in size than the corresponding enzyme; mutant forms of the corresponding enzyme, including but not limited to, naturally-occurring mutants, such as those that vary from the “wild-type” or consensus amino acid sequence, mutants that are generated using physical and/or chemical mutagens, and genetically engineered mutants, for example but not limited to random and site-directed mutagenesis techniques; amino acid insertions and deletions, and changes due to nucleic acid nonsense mutations, missense mutations, and frameshift mutations (see, e.g., Sriskanda and Shuman, Nucl. Acids Res. 26(2):525-31, 1998; Odell et al., Nucl. Acids Res. 31(17):5090-5100, 2003); chimeric enzymes (see, e.g., DNA Amplification: Current Technologies and Applications, Demidov and Broude, eds., Horizon Bioscience, 2004, (“Demidov and Broude”), particularly at chapter 1.1); reversibly modified nucleases, ligases, and extending enzymes, for example but not limited to those described in U.S. Pat. No. 5,773,258; biologically active polypeptides obtained from gene shuffling techniques (see, e.g., U.S. Pat. Nos. 6,319,714 and 6,159,688), splice variants, both naturally occurring and genetically engineered, provided that they are derived, at least in part, from one or more corresponding enzymes; enzymes modified to confer different temperature-sensitive properties (see, e.g., U.S. Pat. Nos. 5,773,258; 5,677,152; and 6,183,998); polypeptides corresponding at least in part to one or more such enzymes that comprise modifications to one or more amino acids of the native sequence, including without limitation, adding, removing or altering glycosylation, disulfide bonds, hydroxyl side chains, and phosphate side chains, or crosslinking, provided such modified polypeptides retain at least some of the desired catalytic activity; and the like. Expressly within the meaning of the term “enzymatically active mutants or variants thereof” when used in reference to a particular enzyme or enzyme type are enzymatically active mutants of that enzyme, enzymatically active variants of that enzyme, or enzymatically active mutants of that enzyme and enzymatically active variants of that enzyme. It is to be understood that when an enzyme or a group of enzymes is recited herein including in the appended claims (for example but not limited to, phi29 DNA polymerase, SP6 RNA polymerase, an extending enzyme, or a ligase) that enzymatically active mutants or variants of that enzyme or type of enzyme are expressly included.

The skilled artisan will readily be able to measure enzymatic activity using an appropriate assay known in the art. Thus, an appropriate assay for polymerase catalytic activity might include, for example, measuring the ability of a variant to incorporate, under appropriate conditions, rNTPs or dNTPs into a nascent polynucleotide strand in a template-dependent manner. Likewise, an appropriate assay for ligase catalytic activity might include, for example, the ability to ligate adjacently hybridized oligonucleotides comprising appropriate reactive groups, such as disclosed herein. Protocols for such assays may be found, among other places, in Sambrook and Russell, Molecular Cloning, A Laboratory Manual, Cold Spring Harbor Press, 3d ed. (2001) (“Sambrook and Russell”); Sambrook, Fritsch, and Maniatis, Molecular Cloning, A Laboratory Manual, Cold Spring Harbor Press, 2d ed. (1989) (“Sambrook et al.”); Ausbel et al., eds., Current Protocols in Molecular Biology, John Wiley & Sons, New York, including updates through December 2004 (“Ausbel et al.”); and Housby and Southern, Nucl. Acids Res. 26:4259-66, 1998).

The term “extending enzyme” refers to a polypeptide that is able to catalyze the 5′-3′ extension of a hybridized primer in template-dependent manner under suitable reaction conditions including without limitation, appropriate nucleotide triphosphates, cofactors, buffer, and the like. Extending enzymes are typically DNA polymerases, for example but not limited to, RNA-dependent DNA polymerases, including without limitation reverse transcriptases, DNA-dependent DNA polymerases, and include DNA polymerases that, at least under certain conditions, share properties of both of these classes of DNA polymerases, and RNA-dependent RNA polymerases. In certain embodiments, an extending enzyme is a reverse transcriptase, for example but not limited to, retroviral reverse transcriptases such as Avian Myeloblastosis Virus (AMV) reverse transcriptase and Moloney Murine Leukemia Virus (MMLV) reverse transcriptase. Certain DNA polymerases possess reverse transcriptase activity under some conditions, for example but not limited to, the DNA polymerase of Thermus thermophilus (Tth DNA polymerase) which demonstrates reverse transcription in the presence of Mn²⁺, but not Mg²⁺ (see also, GeneAmp® AccuRT RNA PCR Kit and Hot Start RNA PCR Kit comprising a recombinant polymerase derived from Thermus specie Z05, both from Applied Biosystems). Likewise, certain reverse transcriptases possess DNA polymerase activity under certain reaction conditions, including without limitation, AMV reverse transcriptase and MMLV reverse transcriptase. Descriptions of extending enzymes can be found in, among other places, Lehninger Principles of Biochemistry, 3d ed., Nelson and Cox, Worth Publishing, New York, N.Y., 2000 (“Lehninger”), particularly Chapters 26 and 29; R. M. Twyman, Advanced Molecular Biology: A Concise Reference. Bios Scientific Publishers, New York, N.Y. (1999); and Enzymatic Resource Guide: Polymerases, Promega, Madison, Wis. (1998).

The terms “hybridizing” and “annealing”, and variations of these terms such as annealed, hybridization, anneal, hybridizes, and so forth, are used interchangeably and mean the nucleotide base-pairing interaction of one nucleic acid with another nucleic acid that results in the formation of a duplex, triplex, or other higher-ordered structure. The primary interaction is typically nucleotide base specific, e.g., A:T, A:U and G:C, by Watson-Crick and Hoogsteen-type hydrogen bonding. In certain embodiments, base-stacking and hydrophobic interactions may also contribute to duplex stability. Conditions under which probes, reporter probes, and primers hybridize to complementary and substantially complementary target sequences, ligated probes, first amplification products, and/or second amplification products are well known in the art, e.g., as described in Nucleic Acid Hybridization, A Practical Approach, B. Hames and S. Higgins, eds., IRL Press, Washington, D.C. (1985) and J. Wetmur and N. Davidson, Mol. Biol. 31:349 et seq. (1968). In general, whether such annealing takes place is influenced by, among other things, the length of the hybridizing region of the probes, primers and reporter probes and their complementary sequences, the pH, the temperature, the presence of mono- and divalent cations, the proportion of G and C nucleotides in the hybridizing region, the viscosity of the medium, and the presence of denaturants. Such variables influence the time required for hybridization. The presence of certain nucleotide analogs or groove binders in the primer or reporter probe can also influence hybridization conditions. Thus, the preferred annealing conditions will depend upon the particular application. Such conditions, however, can be routinely determined by persons of ordinary skill in the art, without undue experimentation.

The term “hybridization tag” as used herein refers to an oligonucleotide sequence that can be used for: separating the element (e.g., ligated probes, first amplification products, second amplification products, surrogates of any of these, including without limitation, ZipChute™ reagents, etc.) of which it is a component or to which it is hybridized, including without limitation, bulk separation; tethering or attaching the element to which it is bound to a capture surface, which may include separating and/or detecting; annealing a corresponding hybridization tag complement; or combinations thereof. In certain embodiments, the same hybridization tag is used with a multiplicity of different elements to effect bulk separation, capture surface attachment, or combinations thereof. In certain embodiments, a hybridization tag provides a unique “address” or identifier to the element containing the hybridization tag. In certain embodiments, this address can be used to identify the corresponding element, for example but not limited to, hybridizing to a particular address or position on an ordered capture surface, including without limitation, a microarray or a bead array, comprising a corresponding hybridization tag complement. In certain embodiments, a primer comprising a unique hybridization tag is incorporated into an amplification product so that the hybridization tag can be subsequently used to bind a reporter probe for detecting that amplification product or its surrogate (see, e.g., U.S. Pat. No. 6,270,967). A “hybridization tag complement” typically refers to an oligonucleotide that comprises a nucleotide sequence that is complementary to at least part of the corresponding hybridization tag. In various embodiments, hybridization tag complements serve as capture moieties for attaching a hybridization tag:element complex to a capture surface for identification, such as multiplex decoding on a microarray, or other purposes; serve as “pull-out” sequences for bulk separation procedures; or both as capture moieties and as pull-out sequences. In certain embodiments, a hybridization tag complement comprises a reporter group, a mobility modifier, a reporter probe-binding portion, or combinations thereof. In certain embodiments, a hybridization tag complement is annealed to a corresponding hybridization tag and, subsequently, at least part of that hybridization tag complement is released and detected. In certain embodiments, detecting comprises a reporter groups on or attached to a hybridization tag complement or at least part of a hybridization tag complement.

Typically, hybridization tags and their corresponding hybridization tag complements are selected to minimize: internal self-hybridization; and cross-hybridization with different hybridization tag species, nucleotide sequences in a sample or reaction composition, including but not limited to target or background sequences, different species of hybridization tag complements, target-specific portions of primers, and the like; but should be amenable to facile hybridization between the hybridization tag and its corresponding hybridization tag complement. Hybridization tag sequences and hybridization tag complement sequences can be selected by any suitable method, for example but not limited to, computer algorithms such as described in PCT Publication Nos. WO 96/12014 and WO 96/41011 and in European Publication No. EP 799,897; and the algorithm and parameters of SantaLucia (Proc. Natl. Acad. Sci. 95:1460-65, 1998). Descriptions of hybridization tags can be found in, among other places, U.S. Pat. Nos. 6,309,829 (referred to as “tag segment” therein); 6,451,525 (referred to as “tag segment” therein); 6,309,829 (referred to as “tag segment” therein); 5,981,176 (referred to as “grid oligonucleotides” therein); 5,935,793 (referred to as “identifier tags” therein); and PCT Publication No. WO 01/92579 (referred to as “addressable support-specific sequences” therein); and Gerry et al., J. Mol. Biol. 292:251-262, 1999) (referred to as “zip-codes” and “zip-code complements” therein). Those in the art will appreciate that a hybridization tag and its corresponding hybridization tag complement are, by definition, complementary to each other and that the terms hybridization tag and hybridization tag complement are relative and can essentially be used interchangeably in most contexts.

Hybridization tags can be located at or near the end of a probe, a primer, an amplification product, or combinations thereof; or they can be located internally. In certain embodiments, a hybridization tag is attached to a probe, a primer, an amplification product, a reporter probe, or combinations thereof, via a linker arm. In certain embodiments, the linker arm is cleavable.

In certain embodiments, hybridization tags are at least 12 bases in length, at least 15 bases in length, 12-60 bases in length, or 15-30 bases in length. In certain embodiments, a hybridization tag is 12, 15, 20, 21, 22, 23, 24, 25, 26, 27, 28, 29, 30, 45, or 60 bases in length. In certain embodiments, at least two hybridization tag:hybridization tag complement duplexes have melting temperatures that fall within a ΔTₘ range (Tₘₐₓ-Tₘᵢₙ) of no more than 10° C. of each other. In certain embodiments, at least two hybridization tag:hybridization tag complement duplexes have melting temperatures that fall within a ΔTₘ range of 5° C. or less of each other.

The term “microorganism” is used in a broad sense and includes non-cellular and unicellular organisms, including colonial organisms, such as eubacteria, including cyanobacteria; archaea; protozoa; fungi, including algae; viruses; and viroids. Exemplary microorganisms include E. coli, including but not limited to enterotoxigenic strains, Staphylococcus species, including but not limited to S. aureus, Streptococcus species, hepatitis A virus, Campylobacter species, Salmonella species, Giardia lamblia, Cryptosporidium species including but not limited to C. parvum and C. muris, rotavirus, Aspergillus species, Bacillus species, including but not limited to B. anthracis, Brucella species, Yersinia pestis, variola major (smallpox virus), Francisella tularensis, and hemorrhagic fever viruses (e.g., filoviruses such as Ebola and Marburg; arenaviruses such as Lassa virus, Machupo virus, etc.), Mycobacterium tuberculosis, Clostridium botulinum, and Francisella tularensis.

The term “mobility modifier” as used herein refers to a molecular entity, for example but not limited to, a polymer chain, that when added to an element (e.g., a probe, a primer, an amplification product, or combinations thereof) affects the mobility of the element to which it is hybridized or bound, covalently or non-covalently, in a mobility-dependent analytical technique. In some embodiments, a mobility modifier changes the charge/translational frictional drag when hybridized or bound to the element; or imparts a distinctive mobility, for example but not limited to, a distinctive elution characteristic in a chromatographic separation medium or a distinctive electrophoretic mobility in a sieving matrix or non-sieving matrix, when hybridized or bound to the corresponding element; or both (see, e.g., U.S. Pat. Nos. 5,470,705 and 5,514,543; Grossman et al., Nucl. Acids Res. 22:4527-34, 1994). In certain embodiments, a multiplicity of different ligated probes and/or amplification products that do not comprise mobility modifiers have the same or substantially the same mobility in a mobility-dependent analytical technique. Typically, such ligated probes and/or amplification products can be separated or substantially separated in a mobility-dependent analytical technique when each such species further comprises an appropriate mobility modifier.

The term “reporter group” is used in a broad sense herein and refers to any identifiable tag, label, or moiety. The skilled artisan will appreciate that many different species of reporter groups can be used in the present teachings, either individually or in combination with one or more different reporter group. The term reporter group also encompasses an element of multi-element indirect reporter systems, including without limitation, affinity tags; and multi-element interacting reporter groups or reporter group pairs, such as fluorescent reporter group-quencher pairs, including without limitation, pairs comprising fluorescent quenchers and dark quenchers, also known as non-fluorescent quenchers (NFQ).

In certain embodiments, a reporter group emits a fluorescent, a chemiluminescent, a bioluminescent, a phosphorescent, or an electrochemiluminescent signal. Exemplary reporter groups include, but are not limited to fluorophores, radioisotopes, chromogens, enzymes, antigens including but not limited to epitope tags, semiconductor nanocrystals such as quantum dots, heavy metals, dyes, phosphorescence groups, chemiluminescent groups, electrochemical detection moieties, affinity tags, binding proteins, phosphors, rare earth chelates, transition metal chelates, near-infrared dyes, including but not limited to, “Cy.7.SPh.NCS,” “Cy.7.OphEt.NCS,” “Cy7.OphEt.CO₂Su”, and IRD800 (see, e.g., J. Flanagan et al., Bioconjug. Chem. 8:751-56 (1997); and DNA Synthesis with IRD800 Phosphoramidite, LI-COR Bulletin #111, LI-COR, Inc., Lincoln, Nebr.), electrochemiluminescence labels, including but not limited to, tris(bipyridal) ruthenium (II), also known as Ru(bpy)₃²⁺, Os(1,10-phenanthroline)₂bis(diphenylphosphino)ethane²⁺, also known as Os(phen)₂(dppene)²⁺, luminol/hydrogen peroxide, Al(hydroxyquinoline-5-sulfonic acid), 9,10-diphenylanthracene-2-sulfonate, and tris(4-vinyl-4′-methyl-2,2′-bipyridal) ruthenium (II), also known as Ru(v-bpy₃²⁺), and the like.

The term reporter group also encompasses an element of multi-element indirect reporter systems, including without limitation, affinity tags such as biotin:avidin, antibody:antigen, ligand:receptor including but not limited to binding proteins and their ligands, and the like, in which one element interacts with one or more other elements of the system in order to effect the potential for a detectable signal. Exemplary multi-element reporter systems include an oligonucleotide comprising a biotin reporter group and a streptavidin-conjugated fluorophore, or vice versa; an oligonucleotide comprising a DNP reporter group and a fluorophore-labeled anti-DNP antibody; and the like. In certain embodiments, reporter groups, particularly multi-element reporter groups, are not necessarily used for detection, but serve as affinity tags for isolation/separation, for example but not limited to, a biotin reporter group and a streptavidin-coated capture surface, or vice versa; a digoxygenin reporter group and a capture surface comprising an anti-digoxygenin antibody or a digoxygenin-binding aptamer; a DNP reporter group and a capture surface comprising an anti-DNP antibody or a DNP-binding aptamer; and the like. Detailed protocols for attaching reporter groups to nucleic acids can be found in, among other places, G. T. Hermanson, Bioconjugate Techniques, Academic Press, San Diego, 1996; Current Protocols in Nucleic Acid Chemistry, S. L. Beaucage et al., eds., John Wiley & Sons, New York, N.Y. (2000), including supplements (“Beaucage”); Handbook of Fluorescent Probes and Research Products, 9^{th} ed., Haugland, Molecular Probes, 2002; and Pierce Applications Handbook and Catalog 2003-2004, Pierce Biotechnology, Rockford, Ill., 2003.

Multi-element interacting reporter groups are also within the scope of the term reporter group, such as fluorophore-quencher pairs, including without limitation fluorescent quenchers and dark quenchers (also known as non-fluorescent quenchers). A fluorescent quencher can absorb the fluorescent signal emitted from a fluorophore and after absorbing enough fluorescent energy, the fluorescent quencher can emit fluorescence at a characteristic wavelength, e.g., fluorescent resonance energy transfer. For example without limitation, the FAM-TAMRA pair can be illuminated at 492 nm, the excitation peak for FAM, and emit fluorescence at 580 nm, the emission peak for TAMRA. A dark quencher, appropriately paired with a fluorescent reporter group, absorbs the fluorescent energy from the fluorophore, but does not itself fluoresce. Rather, the dark quencher dissipates the absorbed energy, typically as heat. Exemplary dark or nonfluorescent quenchers include Dabcyl, Black Hole Quenchers, Iowa Black, QSY-7, AbsoluteQuencher, Eclipse non-fluorescent quencher, metal clusters such as gold nanoparticles, and the like. Certain dual-labeled probes comprising fluorophore-quencher pairs can emit fluorescence when the members of the pair are physically separated, for example but without limitation, nuclease probes such as TaqMan® probes. Other dual-labeled probes comprising fluorophore-quencher pairs can emit fluorescence when the members of the pair are spatially separated, for example but not limited to hybridization probes such as molecular beacons or extension probes such as Scorpion primers. Fluorophore-quencher pairs are well known in the art and used extensively for a variety of reporter probes (see, e.g., Yeung et al., BioTechniques 36:266-75, 2004; Dubertret et al., Nat. Biotech. 19:365-70, 2001; and Tyagi et al., Nat. Biotech. 18:1191-96, 2000).

In certain embodiments, a reporter group comprises an electrochemiluminescent moiety that can, under appropriate conditions, emit detectable electrogenerated chemiluminescence (ECL). In ECL, excitation of the electrochemiluminescent moiety is electrochemically driven and the chemiluminescent emission can be optically detected. Exemplary electrochemiluminescent reporter group species include: Ru(bpy)₃²⁺ and Ru(v-bpy)₃²⁺ with emission wavelengths of 620 nm; Os(phen)₂(dppene)²⁺ with an emission wavelength of 584 nm; luminol/hydrogen peroxide with an emission wavelength of 425 nm; Al(hydroxyquinoline-5-sulfonic acid) with an emission wavelength of 499 nm; and 9,10-diphenylanothracene-2-sulfonate with an emission wavelength of 428 nm; and the like. Forms of these three electrochemiluminescent reporter group species that are modified to be amenable to incorporation into probes and primers are commercially available or can be synthesized without undue experimentation using techniques known in the art. For example, a Ru(bpy)₃²⁺ N-hydroxy succinimide ester for coupling to nucleic acid sequences through an amino linker group has been described (see, U.S. Pat. No. 6,048,687); and succinimide esters of Os(phen)₂(dppene)²⁺ and Al(HQS)₃³⁺ can be synthesized and attached to nucleic acid sequences using similar methods. The Ru(bpy)₃²⁺ electrochemiluminescent reporter group can be synthetically incorporated into nucleic acid sequences using commercially available ru-phosphoramidite (IGEN International, Inc., Gaithersburg, Md.) (see, e.g., Osiowy, J. Clin. Micro. 40:2566-71, 2002).

Additionally other polyaromatic compounds and chelates of ruthenium, osmium, platinum, palladium, and other transition metals have shown electrochemiluminescent properties. Detailed descriptions of ECL and electrochemiluminescent moieties can be found in, among other places, A. Bard and L. Faulkner, Electrochemical Methods, John Wiley & Sons (2001); M. Collinson and M. Wightman, Anal. Chem. 65:2576 (1993); D. Brunce and M. Richter, Anal. Chem. 74:3157 (2002); A. Knight, Trends in Anal. Chem. 18:47 (1999); B. Muegge et al., Anal. Chem. 75:1102 (2003); H. Abrunda et al., J. Amer. Chem. Soc. 104:2641 (1982); K. Maness et al., J. Amer. Chem. Soc. 118:10609 (1996); M. Collinson and R. Wightman, Science 268:1883 et seq. (1995); and U.S. Pat. No. 6,479,233 (see also, O′Sullivan et al., Nucl. Acids Res. 30:e114, 2002 for a discussion of phosphorescent lanthanide and transition metal reporter groups).

As used herein, the term “strand invasion structure” refers to a hybridization complex comprising a double-stranded nucleic acid, for example a double-stranded target sequence, and at least one other component, including without limitation an oligomer comprising PNA, and wherein one strand of the nucleic acid is displaced or “looped out”. In some embodiments, a strand invasion structure comprises a PNA oligomer, including without limitation a pseudocomplementary PNA (pcPNA), for example but not limited to a PNA opener, bis-PNA, or PNA clamp. In some embodiments a strand invasion structure comprises a “P-loop”, also known as (PNA)₂-DNA invasion triplexes, or a “PD-loop”, also known as PNA-distended DNA loops, as generally described in Peptide Nucleic Acids: Protocols and Applications, Neilsen, ed., Horizon Bioscience, 2004, particularly chapters 5 and 10 and Demidov et al., Methods 23:108-122 (see also, Demidov et al., Proc. Natl. Acad. Sci. 99:5953-58, 2002; Kaihatsu et al., Biochem. 41:11118-25, 2002; and Lohse et al., Proc. Natl. Acad. Sci. 96:11804-08, 1999). Typically, the third component of the strand-invasion structure is designed such that the target sequence is looped out, making it accessible for probe hybridization.

The term “surrogate” as used herein refers to any molecule or moiety whose detection or identification indicates the existence of a corresponding ligated probe, a first amplification product, a second amplification product, or combinations thereof, allowing the presence of the corresponding target sequence to be inferred. Exemplary surrogates include but are not limited to, digested amplification products or portions thereof; moieties cleaved or released from an amplification product or amplification product surrogate; complementary strands or counterparts of a amplification product or amplification product surrogate; reporter probes that are or were annealed to a amplification product or another amplification product surrogate, including but not limited to cleavage and amplification products thereof, such as a cleavage fragment of a TaqMan probe or the product of scorpion primer; hybridization tag complements that are or were annealed to a amplification product or another amplification product surrogate, including but not limited to ZipChute™ reagents (typically a molecule or complex comprising a hybridization tag complement, a mobility modifier, and a reporter group, generally a fluorescent reporter group; see, e.g., Applied Biosystems Part Number 4344467 Rev. C; see also U.S. Provisional Patent Application Ser. No. 60/517,470) or parts of hybridization tag complements; detectable luminescence from a chemical and/or enzymatic reaction; and the like. It is to be understood that a second amplification product can serve as a surrogate for the corresponding first amplification product, the corresponding ligated probe, and the corresponding target sequence; that a first amplification product can serve as a surrogate for the corresponding ligated probe and the corresponding target sequence; and that a ligated probe can serve as a surrogate for the corresponding target sequence. Thus, the detection of any of these surrogates, either directly or indirectly, allows the inference that the corresponding target sequence is present in the sample.

The terms “Tm” and “melting temperature” are used interchangeably and refer to the temperature at which a population of double-stranded nucleic acid molecules, including without limitation, a probe-target sequence complex, a first primer-ligated probe complex, a second primer-first amplification product complex, a first primer-second amplification complex, and a double-stranded target sequence, become half (50%) dissociated. Several formulas and computer algorithms for calculating Tm, including chimeric oligomers comprising nucleic acid, are well-known in the art. According to one such predictive formula for oligonucleotides, Tm=(4×number of G+C)+(2×number of A+T). The Tm for a particular oligonucleotide, such as a probe or primer, can also be routinely determined using routine methods, without undue experimentation. Descriptions of melting temperatures and their calculation can be found in, among other places, The Nucleic Acids Protocols Handbook, Rapley, ed., Humana Press, 2000 (“Rapley”); Nielsen, Exiqon Technical Note LNA 02/07.2002, Exiqon A/S; McPherson and Moller, PCR: The Basics, Bios Scientific Publishers, 2000 (“McPherson”); Finn et al., Nucl. Acids Res. 17:3357-63, 1996; and on the internet at, among other places, “appliedbiosystems.com/support/techtools/calc/”, “207.32.43.70/biotools/oligocalc/oligocalc.asp”, and “www-structure.Ilnl.gov/MB_elves/tmcalc.html”.

The term “mobility-dependent analytical technique” as used herein, refers to any means for separating different molecular species based on differential rates of migration of those different molecular species in one or more separation techniques. Exemplary mobility-dependent analytical techniques include electrophoresis, chromatography, sedimentation, e.g., gradient centrifugation, field-flow fractionation, multi-stage extraction techniques and the like. Descriptions of mobility-dependent analytical techniques can be found in, among other places, U.S. Pat. Nos. 5,470,705, 5,514,543, 5,580,732, 5,624,800, and 5,807,682; PCT Publication No. WO 01/92579; D. R. Baker, Capillary Electrophoresis, Wiley-Interscience (1995); Biochromatography: Theory and Practice, M. A. Vijayalakshmi, ed., Taylor & Francis, London, U.K. (2003); Krylov and Dovichi, Anal. Chem. 72:111R-128R (2000); Swinney and Bornhop, Electrophoresis 21:1239-50 (2000); Crabtree et al., Electrophoresis 21:1329-35 (2000); and A. Pingoud et al., Biochemical Methods: A Concise Guide for Students and Researchers, Wiley-VCH Verlag GmbH, Weinheim, Germany (2002).

The term nucleoside refers to a compound comprising a purine nucleotide base, a deazapurine nucleotide base, or a pyrimidine nucleotide base, e.g., adenine (A), guanine (G), cytosine (C), uracil (U), thymine (T), 7-deazaadenine, 7-deazaguanosine, and the like, that is linked to a pentose at the 1′-position. When the nucleotide base is purine or 7-deazapurine, the pentose is attached to the nucleotide base at the 9-position of the purine or deazapurine, and when the nucleotide base is pyrimidine, the pentose is attached to the nucleotide base at the 1-position of the pyrimidine, (e.g., Kornberg and Baker, DNA Replication, 2nd Ed., Freeman, San Francisco, 1992). The term “nucleotide” as used herein refers to a phosphate ester of a nucleoside, e.g., a triphosphate ester, wherein the most common site of esterification is the hydroxyl group attached to the C-5 position of the pentose. As used herein, “nucleotide” refers to a set of compounds including both nucleosides and nucleotides.

The terms “nucleic acid” or “nucleic acid sequence” (including target sequences and non-target sequences) refer to polymers of nucleotide monomers, including analogs of such polymers, including double- and single-stranded deoxyribonucleotides, ribonucleotides, α-anomeric forms thereof, and the like. Monomers are linked by “internucleotide linkages,” e.g., phosphodiester linkages, where as used herein, the term “phosphodiester linkage” refers to phosphodiester bonds or bonds including phosphate analogs thereof, including associated counterions, e.g., H⁺, NH₄⁺, Na⁺, if such counterions are present. Whenever a nucleic acid is represented by a sequence of letters, such as “ATGCCTG,” it will be understood that the nucleotides are in 5′ to 3′ order from left to right, unless otherwise noted or apparent from the context. Nucleic acid sequences typically range in size from a few monomeric units, e.g., 5-90, when they are sometimes referred to as oligonucleotides, to several thousand monomeric nucleotide units or more.

Nucleic acid sequences include without limitation, genomic DNA (gDNA), cDNA, hnRNA, mRNA, rRNA, tRNA, non-coding RNA (ncRNA), fragmented nucleic acid, and nucleic acid obtained from subcellular organelles such as mitochondria. Synthetic target sequences that are “spiked” into a sample or reaction composition, including “control sequences” or “standards” that can be used for, among other things, standardizing and/or validating the performance of a disclosed method or kit, are also within the scope of the current teachings.

The term “analogs” when used in reference to nucleotides and/or nucleic acid sequences comprise synthetic analogs having modified nucleotide base portions, modified pentose portions and/or modified phosphate portions, and, in the case of polynucleotides, modified internucleotide linkages, as described generally elsewhere (e.g., Scheit, Nucleotide Analogs, John Wiley, New York, 1980; Englisch, Angew. Chem. Int. Ed. Engl. 30:613-29, 1991; Agarwal, Protocols for Polynucleotides and Analogs, Humana Press, 1994; and S. Verma and F. Eckstein, Ann. Rev. Biochem. 67:99-134, 1998). Generally, modified phosphate portions comprise analogs of phosphate wherein the phosphorous atom is in the +5 oxidation state and one or more of the oxygen atoms are replaced with a non-oxygen moiety, e.g., sulfur. Exemplary phosphate analogs include but are not limited to phosphorothioate, phosphorodithioate, phosphoroselenoate, phosphorodiselenoate, phosphoroanilothioate, phosphoranilidate, phosphoramidate, boronophosphates, including associated counterions, e.g., H⁺, NH₄⁺, Na⁺, if such counterions are present. Exemplary modified nucleotide base portions include but are not limited to 2,6-diaminopurine, hypoxanthine, pseudouridine, C-5-propyne, isocytosine, isoguanine, 2-thiopyrimidine, and other like analogs. Particularly preferred nucleotide base analogs are iso-C and iso-G nucleobase analogs available from Sulfonics, Inc., Alachua, Fla. (e.g., Benner, et al., U.S. Pat. No. 5,432,272) or locked nucleic acid (LNA) analogs (e.g., Koshkin et al., Tetrahedron 54:3607-30, 1998). Exemplary modified pentose portions include but are not limited to 2′- or 3′-modifications where the 2′- or 3′-position is hydrogen, hydroxy, alkoxy, e.g., methoxy, ethoxy, allyloxy, isopropoxy, butoxy, isobutoxy and phenoxy, azido, amino or alkylamino, fluoro, chloro, bromo and the like. Modified internucleotide linkages include phosphate analogs, analogs having achiral and uncharged intersubunit linkages (e.g., Sterchak et al., Organic Chem. 52:4202, 1987), and uncharged morpholino-based polymers having achiral intersubunit linkages (e.g., U.S. Pat. No. 5,034,506). Some internucleotide linkage analogs include morpholidate, acetal, and polyamide-linked heterocycles. In one class of nucleotide analogs, known as peptide nucleic acids, including pseudocomplementary peptide nucleic acids (collectively “PNA”), a conventional sugar and internucleotide linkage has been replaced with a 2-aminoethylglycine amide backbone polymer (see, e.g., Nielsen et al., Science, 254:1497-1500, 1991; Egholm et al., J. Am. Chem. Soc., 114: 1895-1897 1992; Demidov et al., Proc. Natl. Acad. Sci. 99:5953-58, 2002; Peptide Nucleic Acids: Protocols and Applications, Nielsen, ed., Horizon Bioscience, 2004). Descriptions of oligonucleotide synthesis and analogs, can be found in, among other places, S. Verma and F. Eckstein, Ann. Rev. Biochem. 67:99-134 (1999); J. Goodchild, Bioconj. Chem. 1:165-87 (1990); Beaucage; Nucleic Acids in Chemistry and Biology, 2d ed., Blackburn and Gait, eds., Oxford University Press, 1996; and U.S. Pat. Nos. 4,373,071; 4,401,796; 4,415,732; 4,458,066; 4,500,707; 4,668,777; 4,973,679; 5,047,524; 5,132,418; 5,153,319; and 5,262,530. It is to be understood that terms such as “three of the four nucleotide bases: (1) A, (2) C, (3) G, and (4) T, U, or T and U, but not the fourth nucleotide base” and similar terms, as used herein including in the appended claims, can encompass analogs.

The terms “universal base” or “universal nucleotide” are generally used interchangeably herein and refer to a nucleotide analog that can substitute for more than one of the natural nucleotides or natural bases in oligonucleotides. Universal bases typically contain an aromatic ring moiety that may or may not contain nitrogen atoms and generally use aromatic ring stacking to stabilize a duplex. Some universal bases can be covalently attached to the C-1′ carbon of a pentose sugar to make a universal nucleotide. Some universal bases do not hydrogen bond specifically with another nucleotide base. Some universal bases may interact with adjacent nucleotide bases on the same nucleic acid strand by hydrophobic stacking. Exemplary universal bases include deoxy-7-azaindole triphosphate (d7AITP), deoxyisocarbostyril triphosphate (dICSTP), deoxypropynylisocarbostyril triphosphate (dPICSTP), deoxymethyl-7-azaindole triphosphate (dM7AITP), deoxylmPy triphosphate (dlmPyTP), deoxyPP triphosphate (dPPTP), deoxypropynyl-7-azaindole triphosphate (dP7AITP), 3-methyl isocarbostyril (MICS), 5-methyl isocarbyl (5MICS), imidazole-4-carboxamide, 3-nitropyrrole, 5-nitroindole, hypoxanthine, inosine, deoxyinosine, 5-fluorodeoxyuridine, and 4-nitrobenzimidizole. Detailed descriptions of universal bases can be found in, among other places, Loakes, Nucl. Acids Res. 29:2437-47, 2001; Berger et al., Nucl. Acids Res. 28:2911-14, 2000; Loakes et al., J. Mol. Biol. 270:426-35, 1997; Verma and Eckstein, Ann. Rev. Biochem. 67:99-134, 1998; Published PCT Application No. US02/33619, and U.S. Pat. Nos. 6,433,134 and 6,433,134. Those in the art will appreciate that universal bases can typically be incorporated into oligonucleotide probes using conventional synthesis techniques.

### II. REAGENTS

The term “target sequence” refers to the nucleic acid sequence being selectively amplified or being detected. Often a target sequence is selected because it is a diagnostic indicator for a particular microorganism, related species of microorganisms, a particular cell type in a tissue or clinical sample, including without limitation, malignant cells in a biopsy specimen, a human identity marker in a forensic sample, and the like. Target sequences typically constitute only a fraction of the total nucleic acid composition of a sample being analyzed, which can make their amplification and/or detection by conventional methodology problematic.

According to the current teachings, a target sequence can be derived from any living, or once living, organism, including but not limited to prokaryote, eukaryote, plant, animal, and virus. The target sequence may originate from a nucleus of a cell, e.g., genomic DNA, or may be extranuclear nucleic acid, e.g., plasmid, mitrochondrial nucleic acid, various RNAs, and the like. The target nucleic acid sequence may be first reverse-transcribed into cDNA if the target nucleic acid is RNA. Furthermore, the target sequence may be present in a double-stranded or single-stranded form.

A variety of well-known techniques are available for obtaining a target sequence for use with the disclosed methods. When the target sequence is obtained through isolation from a biological matrix, preferred isolation techniques include (1) organic extraction followed by ethanol precipitation, e.g., using a phenol/chloroform organic reagent (e.g., Ausbel et al.), preferably using an automated DNA extractor, e.g., the Model 341 DNA Extractor available from Applied Biosystems (Foster City, Calif.); (2) stationary phase adsorption methods (e.g., U.S. Pat. No. 5,234,809; Walsh et al., Biotechniques 10:506-513, 1991); and (3) salt-induced DNA precipitation methods (e.g., Miller et al., Nucleic Acids Research, 16(3):9-10, 1988), such precipitation methods being typically referred to as “salting-out” methods. Optimally, each of the above isolation methods is preceded by an enzyme digestion step to help eliminate unwanted protein from the sample, e.g., digestion with proteinase K, or other like proteases (see, e.g., U.S. patent application Ser. No. 10/618,493). Those in the art understand that a number of sample preparation kits and instruments are commercially available, including without limitation, ABI Prism TransPrep System, BloodPrep Chemistry, NucPrep Chemistry, PrepMan Ultra Sample Preparation Reagent, ABI Prism 6100 Nucleic Acid PrepStation, and ABI Prism 6700 Automated Nucleic Acid Workstation (all from Applied Biosystems).

In some embodiments, the target sequence is from a microorganism. In other embodiments, the target sequence is from a mammal, such as a human, and the sequence is typically an indicator of a particular condition or physiological state in that mammal, for example but not limited to, cancer or a genetic disorder. In certain circumstances, a human target sequence is selectively amplified and/or detected for forensics evaluation or human identification.

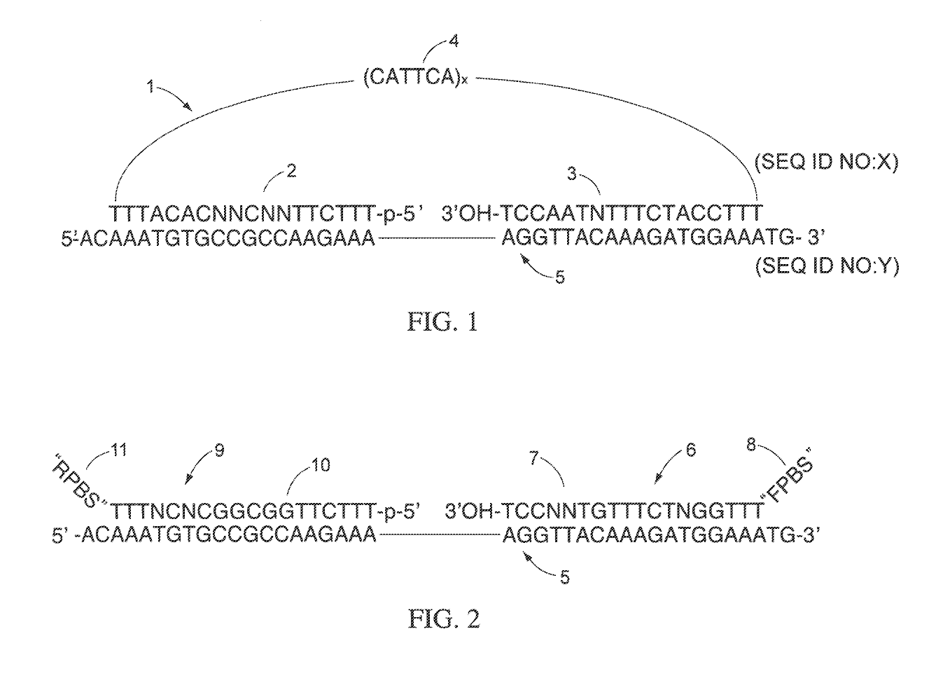

In some embodiments, circularizable target-specific probes are provided (see, e.g., FIG. 1). Such probes typically comprise: (a) a first target-complementary portion, (b) a second target-complementary portion, and (c) a spacer portion. The probe's first target-complementary portion comprises three of the four nucleotide bases: (1) A, (2) C, (3) G, and (4) T, U, or T and U, but not the fourth nucleotide base. These nucleotide bases are typically in the form of deoxyribonucleotides, ribonucleotides, or combinations thereof. The three nucleotide bases that are present in the first target-complementary portion are also typically present in the second target-complementary portion and the spacer portion, but the fourth nucleotide base that is absent from the first target-complementary portion is not present. For illustration purposes but not as a limitation, one exemplary probe of the current teachings contains the nucleotide bases A, C, and G, but not the nucleotide base T, U, or T and U; another exemplary probe includes the nucleotide bases (i) C, G, and U, (ii) C, G, and T, or (iii) C, G, T and U, but not the nucleotide base A; and so forth. In certain embodiments, the disclosed probes consist of three nucleotide bases, but not the fourth nucleotide base; in other embodiments, the target-specific probes consist essentially of three of the nucleotide bases, but not the fourth nucleotide base.

Those in the art will appreciate that the nucleotide bases T and U can generally be used interchangeably. For example, the sequences TACTAC, UACTAC, and UACUAC are generally viewed as essentially equivalent and for purposes of the current teachings, “T, U, or T and U” are to be considered one nucleotide base. Those in the art will also understand that nucleotide base analogs, for example but not limited to 5-methylcytosine as an analog for cytosine, are within the contemplation of the present teachings.

In some embodiments, a pair of target-specific probes (also referred to as a “probe pair”) is provided, wherein each probe pair comprises a first probe and a corresponding second probe (see, e.g., FIG. 2). The probes of each probe pair is designed to hybridize with corresponding regions of the same target sequence, with the first probe comprising a first target-complementary portion and the second probe comprising a second target-complementary portion. The two target-complementary portions of each probe pair comprise three of the four nucleotide bases: (1) A, (2) C, (3) G, and (4) T, U, or T and U, but not the fourth, and both target-complementary portions include the same three nucleotide bases, but not the fourth nucleotide base. Typically any additional nucleotides in the probe pair also comprise the same three nucleotide bases, but not the fourth. In some embodiments, the first target-complementary portion of the first probe, the second target-complementary portion of the corresponding second probe, or both the first target-complementary portion of the first probe and the second target-complementary portion of the corresponding second probe comprises a universal base. In some embodiments, a probe comprising a target-complementary portion comprising three of the four nucleotide bases: (1) A, (2) C, (3) G, and (4) T, U, or T and U, but not the fourth nucleotide base is provided.

In some embodiments, a circularizable probe and/or a probe pair consists of three of the four nucleotide bases: (1) A, (2) C, (3) G, and (4) T, U, or T and U, but not the fourth nucleotide base. In other embodiments, a circularizable probe and/or a probe pair consists of a universal base and three of the four nucleotide bases: (1) A, (2) C, (3) G, and (4) T, U, or T and U, but not the fourth nucleotide base. In other embodiments, a circularizable probe and/or a probe pair consists essentially of three of the four nucleotide bases: (1) A, (2) C, (3) G, and (4) T, U, or T and U, but not the fourth nucleotide base. In other embodiments, a circularizable probe and/or a probe pair consists essentially of a universal base and three of the four nucleotide bases: (1) A, (2) C, (3) G, and (4) T, U, or T and U, but not the fourth nucleotide base. In some embodiments, the additional nucleotide bases in the probe(s), if any, comprise, consist essentially of, or consist of the same three nucleotide bases as the target-complementary portion(s), but not the fourth nucleotide base.

Those in the art understand that any of a number of universal bases can be incorporated into the probes and probe sets of the current teachings and that the same universal base can be used throughout or that different universal bases may be used, provided that the resulting probe or probe set can specifically hybridize with the corresponding target sequence under the reaction conditions employed. It is to be understood that the spacer can contain a variable number of nucleotides, depending at least in part on the length of the respective target-complementary portions, the application, and functionality of the probe, and that the spacer can, but need not contain repeat sequences.

Those in the art will also appreciate that various combinations of three nucleotide bases may be employed in the disclosed probes and/or probe pairs, based at least in part on the target sequence and the reaction conditions. It is to be understood that the Tm of different probes may be different from each other, but their respective Tm can be predicted using well known formulas/algorithms or determined using routine methods; and that one or more appropriate nucleotide base combinations can be obtained for use in a desired application. General information on probe and primer design can be found in, among other places, Diffenbach and Dveksler, PCR Primer, A Laboratory Manual, Cold Spring Harbor Press, 1995; Rapley; McPherson; and Kwok et al., Nucl. Acid Res. 18:999-1005, 1990. The target-complementary portion(s) of the disclosed probe(s) and the sequence-specific portions of primers are sufficiently long to permit specific annealing to complementary sequences in target sequences, ligated probes, first amplification products, and second amplification products, as appropriate.

In some embodiments, the universal base in the target-complementary portion of a disclosed probe or probe pair is incorporated in place of the missing fourth nucleotide base (e.g., FIGS. 1 and 2). In some embodiments, a universal base is included to broaden the probe's specificity, for example to allow a probe to hybridize with different, but closely related target sequences, such as different strains of a bacterial species, related species of the same genus of microorganism, or alternate alleles of an irrelevant SNP site within the target sequence. In some embodiments, universal bases are used both to replace the absent fourth nucleotide base and to broaden probe specificity. In some embodiments, all of the universal bases in a probe or a probe set are the same, while in other embodiments at least two different universal bases are incorporated into the probe or probe set. In some embodiments, a universal base is incorporated in place of the least frequently occurring of the four nucleotide bases in the complement of the target sequence.

The term “primer”, when used in reference to a first primer or a second primer, means an oligonucleotide or chimeric oligomer that hybridizes with a corresponding ligated probe, a first amplification product, a second amplification product, or combinations thereof. A primer is an oligonucleotide or chimeric oligomer that is capable of acting as an initiation point for primer extension reactions and when extended, the primer typically becomes incorporated into the resulting amplification product. According to the present teachings, a first primer is designed to hybridize with a corresponding ligated probe and in some embodiments, a corresponding second amplification product; a second primer is designed to hybridize with a corresponding first amplification product. In certain embodiments, a first primer, a second primer, or both, further comprise a universal priming sequence, including without limitation, a primer-binding site for a universal primer. A “universal primer” is a sequence that is designed to hybridize with and prime the amplification of more than one species of ligated probe, amplification product, or combinations thereof.

The probes, probe sets, primers and other synthetic sequences, including certain chimeric oligomers of the current teachings, can be synthesized by any conventional methodology, including without limitation, chemical synthesis techniques for example, phosphoroamidite chemistry and nucleic acid synthesizers such as the Expedite 8900 Nucleic Acid Synthesis System (Applied Biosystems) or enzymatic means, including without limitation, DNA polymerase, RNA polymerase, or reverse transcriptase (see, e.g., Myer and Day, BioTechniques 30:584-93, 2001; Rapley; Ausbel et al.; Finn et al., Nucl. Acids Res. 17:3357-63, 1996; Loakes and Brown, Nucl. Acids Res. 22:4039-43, 1994; Ohtsuka et al., J. Biol. Chem. 260:2605-08, 1985; Peptide Nucleic Acids: Protocols and Applications, Nielsen and Egholm, eds., Horizon Scientific Press, 1999, particularly at Chapter 2.4; and Braasch and Corey, Methods 23:97-107, 2001). Probes can be made suitable for ligation during synthesis or by a post-synthesis modification technique, as appropriate. It is to be understood that the nucleic acid and chimeric oligomer synthesis technique is generally not limiting. In certain embodiments, a circularizable probe, a first probe of a probe pair, a second probe of a probe pair, a first primer, a second primer, or combinations thereof, comprise a primer binding-site, a reporter probe-binding site, a promoter sequence, a reporter group, a mobility modifier, an affinity tag, a hybridization tag, a ribosome-binding site, or combinations thereof.

According to the present teachings, the term “extending enzyme” refers to a polypeptide that is able to catalyze the 5′-3′ extension of a hybridized primer in template-dependent manner under suitable reaction conditions including without limitation, appropriate nucleotide triphosphates, cofactors, buffer, and the like. Exemplary extending enzymes include DNA-directed DNA polymerases, such as Taq DNA polymerase, bacteriophage phi29 DNA polymerase, Bst DNA polymerase, and Tth DNA polymerase; RNA-directed DNA polymerases, including without limitation reverse transcriptases, such as AMV reverse transcriptase; and RNA-dependent RNA polymerases, such as bacteriophage T3, SP6, or T7 RNA polymerase. In certain embodiments, an extending enzyme is thermostable, such as Taq DNA polymerase. Descriptions of extending enzymes can be found in, among other places, Lehninger Principles of Biochemistry, 3d ed., Nelson and Cox, Worth Publishing, New York, N.Y., 2000 (“Lehninger”), particularly Chapters 26 and 29; R. M. Twyman, Advanced Molecular Biology: A Concise Reference. Bios Scientific Publishers, New York, N.Y. (1999); and Enzymatic Resource Guide: Polymerases, Promega, Madison, Wis. (1998).

The term “ligation agent” or “ligation means” according to the present teachings comprises any number of enzymatic or non-enzymatic agents that can effect ligation of nucleic acids to one another, including without limitation, ligases, chemical ligation agents and photoligation. For example, ligase is an enzymatic ligation agent that, under appropriate conditions, forms phosphodiester bonds between the 3′-OH and the 5′-phosphate of adjacent probes or the ends of a circularizable probe. Temperature sensitive ligases, include but are not limited to, bacteriophage T4 ligase and E. coli ligase. Exemplary thermostable ligases include, without limitation, Afu ligase, Taq ligase, Tfl ligase, Mth ligase, Tth ligase, Tth HB8 ligase, Tsc ligase, Thermus species AK16D ligase, Ape ligase, Lig_{Tk} ligase, Aae ligase, Rm ligase, and Pfu ligase (see, e.g., Housby et al., Nucl. Acids Res. 28:e10, 2000; Tong et al., Nucl. Acids Res. 28:1447-54, 2000; Nakatani et al., Eur, J. Biochem. 269:650-56, 2002; and Sriskanda et al., Nucl. Acids Res. 11:2221-28, 2000). The skilled artisan will appreciate that any number of thermostable ligases, including DNA ligases and RNA ligases, can be obtained from thermophilic or hyperthermophilic organisms, for example, certain species of eubacteria and archaea, including viruses that infect such thermophilic or hyperthermophilic organisms; and that such ligases can be employed in the disclosed methods and kits.

Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the teachings herein. Protocols for chemical ligation methods and descriptions of appropriate reactive groups can be found in, among other places, Xu et al., Nucl. Acids Res., 27:875-81, 1999; Gryaznov and Letsinger, Nucl. Acids Res. 21:1403-08, 1993; Gryaznov et al., Nucleic Acid Res. 22:2366-69, 1994; Kanaya and Yanagawa, Biochemistry 25:7423-30, 1986; Luebke and Dervan, Nucl. Acids Res. 20:3005-09, 1992; Sievers and von Kiedrowski, Nature 369:221-24, 1994; Liu and Taylor, Nucl. Acids Res. 26:3300-04, 1999; Wang and Kool, Nucl. Acids Res. 22:2326-33, 1994; Purmal et al., Nucl. Acids Res. 20:3713-19, 1992; Ashley and Kushlan, Biochemistry 30:2927-33, 1991; Chu and Orgel, Nucl. Acids Res. 16:3671-91, 1988; Sokolova et al., FEBS Letters 232:153-55, 1988; Naylor and Gilham, Biochemistry 5:2722-28, 1966; James and Ellington, Chem. & Biol. 4:595-605, 1997; and U.S. Pat. No. 5,476,930.

Photoligation using light of an appropriate wavelength as a ligation agent is also within the scope of the current teachings. In certain embodiments, photoligation comprises probes comprising nucleotide analogs, including but not limited to, 4-thiothymidine (s⁴T), 5-vinyluracil and its derivatives, or combinations thereof. In certain embodiments, the ligation agent comprises: (a) light in the UV-A range (about 320 nm to about 400 nm), the UV-B range (about 290 nm to about 320 nm), or combinations thereof, (b) light with a wavelength between about 300 nm and about 375 nm, (c) light with a wavelength of about 360 nm to about 370 nm; (d) light with a wavelength of about 364 nm to about 368 nm, or (e) light with a wavelength of about 366 nm. In certain embodiments, photoligation is reversible. Descriptions of photoligation can be found in, among other places, Fujimoto et al., Nucl. Acid Symp. Ser. 42:39-40, 1999; Fujimoto et al., Nucl. Acid Res. Suppl. 1:185-86, 2001; Fujimoto et al., Nucl. Acid Suppl., 2:155-56, 2002; Liu and Taylor, Nucl. Acid Res. 26:3300-04, 1998; and on the world wide web at: sbchem.kyoto-u.ac.jp/saito-lab.

When used in the context of the current teachings, the term “suitable for ligation” refers to one or more ends of a circularizable probe or one or more ends of a probe of a probe set that comprises an appropriately reactive group for a particular ligation agent. For example but not limited to, the 3′- and 5′-ends of a circularizable probe or the ends or two corresponding probes of a probe set. Exemplary pairs of reactive groups include, but are not limited to: a nucleotide 3′-hydroxyl group on the 3′ end of a probe and a nucleotide 5′-phosphate group on the 5′ end of the same or corresponding second probe; phosphorothioate and tosylate or iodide; esters and hydrazide; RC(O)S⁻, haloalkyl, or RCH₂S and α-haloacyl; thiophosphoryl and bromoacetoamido groups. Additionally, in certain embodiments, the 5′-end and the 3′-end of a target-specific probe are hybridized adjacently on the target sequence to allow ligation. In other embodiments, the 5′-end and the 3′-end of the probe are not immediately adjacent when they hybridize and a gap-filling step is employed to extend the 5′-end of the probe into juxtaposition with the 3′-end of the probe. In yet other embodiments, there is a gap between the probe's 5′-end and its 3′-end such that a “gap oligonucleotide” can hybridize in the gap between the two ends, for example, to increase specificity. In such embodiments, the 5′-end and the 3′-end of the probe can be ligated to the 3′-end and the 5′-end of the gap oligonucleotide, respectively. It is to be understood that, according to the current teachings, gap nucleotides are designed to include three of the four nucleotide bases, but not the fourth, and potentially a universal base, consistent with the probe or probe set being employed; and that gap-filling includes incorporation of three of the four nucleotide bases, but not the fourth, and potentially a universal base, consistent with the probe or probe set being employed.

Luminescence, used in a broad sense, refers to any process by which light is generated without raising the temperature significantly, including without limitation, bioluminescence, chemiluminescence, phosphorescence, electrochemiluminescence, and fluorescence. As used herein, the term “luminescence-generating means” refers to any method for generating luminescence or light, including without limitation bioluminescence or chemiluminescence methods employing an enzymatic or chemical reaction, respectively. Exemplary luminescence-generating means include the enzyme luciferase derived from any species, for example but not limited to, firefly luiciferase, Renilla luciferase, Gaussia princeps luciferase, Pleuromamma luciferase, and its substrate, typically luciferin or coelenterazine; luminol chemiluminescence; peroxyoxalates, such as bis(2,4,6-trichlorophenyl)oxalate (TCPO) and hydrogen peroxide; luminal and hydrogen peroxide with potassium hexacyanoferrate as a catalyst. Descriptions of luminescence and luminescence-generating means can be found in, among other places, Methods of Enzymology, Vol. 133, DeLuca and McElroy, eds., Academic Press, 1986; and Methods of Enzymology, Vol. 305, Ziegler and Baldwin, eds., Academic Press, San Diego, 2000.

The term “reporter probe” refers to a sequence of nucleotides, nucleotide analogs, or nucleotides and nucleotide analogs, that binds to or anneals with a first amplification product, a second amplification product, an amplification product surrogate, or combinations thereof, and when detected, including but not limited to a change in intensity or of emitted wavelength, is used to identify the corresponding target sequence. Most reporter probes can be categorized based on their mode of action, for example but not limited to: nuclease probes, including without limitation TaqMan® probes (see, e.g., Livak, Genetic Analysis Biomolecular Engineering 14:143-149, 1999; Yeung et al., BioTechniques 36:266-75, 2004); extension probes such as scorpion primers, Lux™ primers, Amplifluors, and the like; hybridization probes such as molecular beacons, Eclipse probes, light-up probes, pairs of singly-labeled reporter probes, hybridization probe pairs, or combinations thereof. In certain embodiments, reporter probes comprise an amide bond, an LNA, a universal base, or combinations thereof, and include stem-loop and stem-less reporter probe configurations. Certain reporter probes are singly-labeled, while other reporter probes are doubly-labeled. Dual probe systems that employ fluorescence resonance energy transfer (FRET) between adjacently hybridized probes are within the intended scope of the term reporter probe.

In certain embodiments, a reporter probe comprises a fluorescent reporter group, a quencher reporter group (including without limitation dark quenchers and fluorescent quenchers), an affinity tag, a hybridization tag, a hybridization tag complement, or combinations thereof. In certain embodiments, a reporter probe comprising a hybridization tag complement anneals with the corresponding hybridization tag, a member of a multi-component reporter group binds to a reporter probe comprising the corresponding member of the multi-component reporter group, or combinations thereof. Exemplary reporter probes include TaqMan® probes; Scorpion probes (also referred to as scorpion primers); Lux™ primers; FRET primers; Eclipse probes; hybridization probe pairs; molecular beacons, including but not limited to FRET-based molecular beacons, multicolor molecular beacons, aptamer beacons, PNA beacons, and antibody beacons; reporter group-labeled PNA clamps, reporter group-labeled PNA openers, reporter group-labeled LNA probes, and probes comprising nanocrystals, metallic nanoparticles and similar hybrid probes (see, e.g., Tyagi and Kramer, Nature Biotech. 14:303-08, 1995; Nazarenko et al., Nucl. Acids Res. 25:2516-21, 1997; Fiandaca et al., Genome Res. 11:609-13, 2001; Dubertret et al., Nature Biotech. 19:365-70, 2001; Zelphati et al., BioTechniques 28:304-15, 2000; and Wilhelm and Pingoud, Chem Bio Chem 2:1120-28, 2003). In certain embodiments, reporter probes further comprise groove binders including but not limited to TaqMan®MGB probes and TaqMan®MGB-NFQ probes (both from Applied Biosystems). In certain embodiments, reporter probe detection comprises fluorescence polarization detection (see, e.g., Simeonov and Nikiforov, Nucl. Acids Res. 30:e91, 2002).

### III. TECHNIQUES

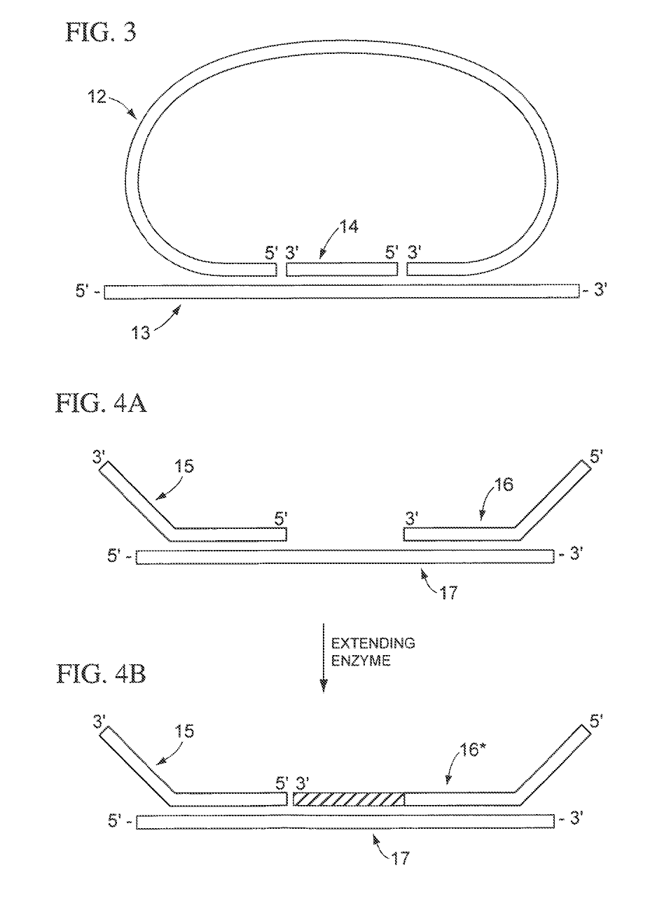

According to the current teachings, methods for selectively amplifying a target sequence and methods for detecting a target sequence are provided. In some embodiments, a probe of the current teachings is hybridized with the target sequence via the probe's or probe pair's first- and second-target complementary portions. In some embodiments, the 5′-end and the 3′-end of the probe(s) are immediately adjacent when hybridized to the target sequence (see, e.g., FIGS. 1 and 2). In other embodiments, there is initially a gap or space between the ends of the hybridized probe(s) that is filled using, for example, a gap oligonucleotide or a “gap-filling” technique known in the art (see, e.g., Lizardi et al., Nat. Genetics 19:225-32, 1998). In one example, depicted schematically in FIG. 3, probe 12 hybridizes with target sequence 13 with a space between the ends of the probe, such that the corresponding “gap oligonucleotide” 14 can also hybridize with target sequence 13. The probe's 5′- and 3′-ends are adjacent to the 3′- and 5′-end of the gap oligonucleotide, respectively. In another exemplary embodiment, depicted schematically in FIG. 4, there is a space between the opposing ends of the first probe 15 and second probe 16 of the illustrative probe pair when the two probes initially hybridize to the target sequence 17 (FIG. 4A). The 3′-end of second probe 16 can be extended, for example by “gap-filling” using an extending enzyme, so that the newly synthesized 3′-end of the extended probe 16* is adjacent to the 5′-end of first probe 15, (FIG. 4B; the “gap-filled” sequence is shown shaded).

The ends of the hybridized probe(s), and where appropriate, the gap oligonucleotide, can be ligated together to form a ligated probe-target complex, provided that the respective ends are suitable for ligation. In some embodiments, an enzymatic ligation means is used. In some embodiments, non-enzymatic ligation means are employed, such as chemical or photoligation means. In some embodiments, the ligated probe is displaced from the target sequence, for example but not limited to, thermal denaturation; chemical denaturation, including without limitation, basic pH (e.g. sodium hydroxide, ammonium hydroxide), formamide, urea, DMSO (to some extend), and cation chelating agents; helicase; or the formation of a strand invasion structure, for example but not limited to, certain PNA and/or pcPNA molecules complementary to at least a part of the target sequence, or combinations thereof. In other embodiments, the ligated probe is released or at least partially displaced by a first primer, wherein the first primer comprises a chimeric oligomer, including without limitation, a DNA-PNA chimera or a DNA-LNA chimera. Those in the art will appreciate that many chimeric first primer oligomers are possible, provided that they (i) are able to hybridize with the ligated probe and in so doing displaces the ligated probe at least partially from the ligated probe-target complex, and (ii) are extendable by an extending enzyme. In some embodiments, a oligomer, including without limitation a PNA oligomer or chimeric oligomer, hybridizes with a portion of the target sequence of a ligated probe-target sequence complex to release or at least partially displace the ligated probe and subsequently, a first primer binds to the ligated probe. In some embodiments, the ligated probe is not displaced prior to, or as the result of, first primer hybridization.

A first primer is hybridized to the ligated probe and the hybridized primer is extended in a first reaction composition to generate a first amplification product. Typically, the first primer hybridizes to the ligated probe at or near the ligation site on the upstream side, so that only ligated probes, not unligated probes are amplified. The first reaction composition is nucleotide-deficient, in that only three of the four nucleotide bases are present, typically in the form of nucleotide triphosphates, wherein the three nucleotide bases in the reaction composition are complementary with the nucleotide bases in the ligated probe. Those in the art will appreciate that because one of the four NTPs is absent from the first reaction composition, background sequences that include all four nucleotide bases are not amplified, even if mispriming occurs (although some limited, abortive amplification is possible). However, ligated probes containing only three nucleotide bases (and optionally a universal base) can be amplified in appropriate first reaction compositions. Thus, the ligated probes but not the background sequences are selectively amplified, even if mispriming occurs, because the nucleotide-deficient reaction composition will not support the amplification of the background sequences but will support the amplification of appropriate ligated probes. Those in the art will understand that ligated probes comprising a universal base, including without limitation, different universal bases, can also be selectively amplified in appropriate nucleotide-deficient first reaction compositions of the present teachings.

In certain embodiments, a second primer is hybridized with the first amplification product and amplified to generate a second amplification product. In some embodiments, the first amplification product is separated from at least some of the non-target sequences in the first reaction composition before the second amplification product is generated. In certain embodiments, a second reaction composition is formed comprising second primers and all four nucleotide bases (i.e., A, C, G, and T, U, or T and U) and second amplification products generated using the first amplification products as templates. In some embodiments, the second reaction composition comprises first primers and the second amplification products serve as templates for generating additional first amplification products.

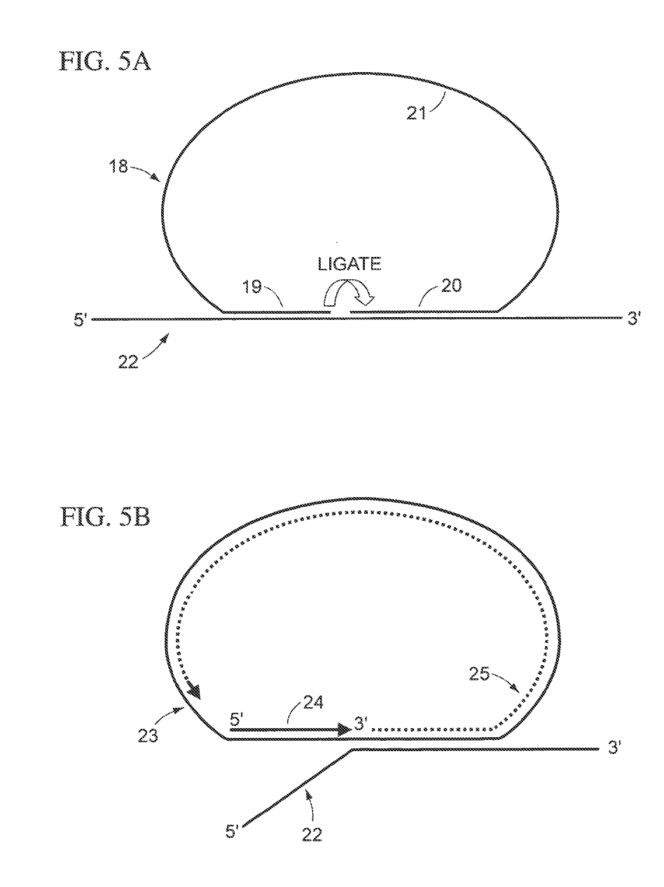

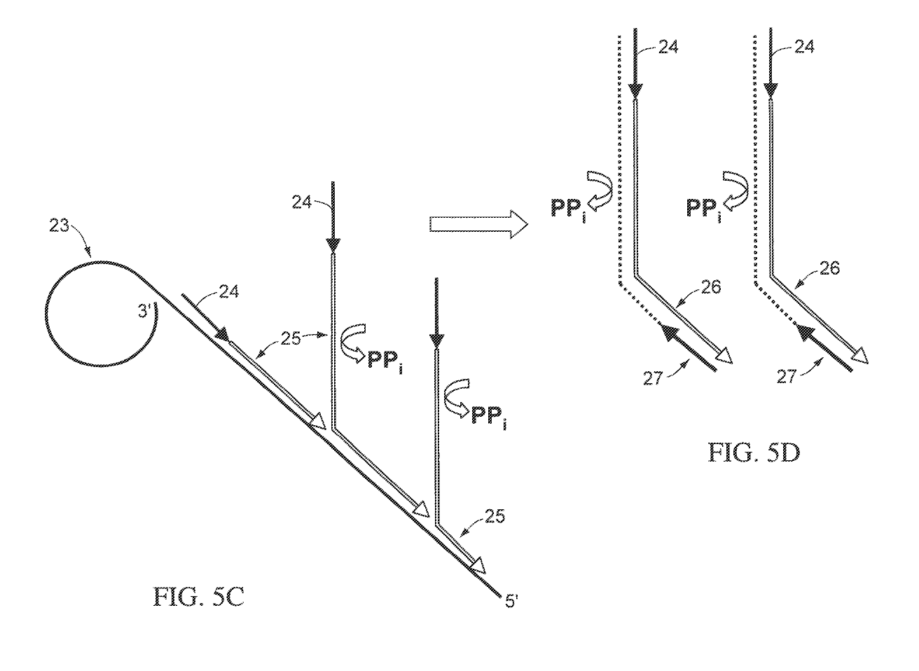

One exemplary embodiment comprising rolling circle amplification is depicted in FIGS. 5A-D. As shown in FIG. 5A, a circularizable probe 18, comprising a first target-complementary portion 19, a second target-complementary portion 20, and a spacer 21, hybridizes with a target sequence 22 and the probe's 5′-end is ligated with its 3′-end to generate a ligated probe 23 (see FIG. 5B). As shown in FIG. 5B, a first primer 24 hybridizes with ligated probe 23, displacing target sequence 22. In some embodiments of the disclosed methods, the ligation product-target complex is released prior to primer hybridization by, for example but not limited to, thermal or chemical denaturation. Returning to FIG. 5B, in a first reaction composition the hybridized first primer 24 is selectively amplified by a first extending enzyme in a template-dependent manner to generate a first amplification product 25. Provided that the first extending enzyme has strong strand displacement activity, rolling circle amplification (RCA) can occur. As shown in FIG. 5C, when the first primer-binding sites are displaced from ligated probe 23 by RCA, additional copies of first primer 24 hybridize and are extended by an extending enzyme to generate additional first amplification products 25. Inorganic pyrophosphate (shown as “PPi”) is generated as a product of the polymerization reaction. In some embodiments, the PPi serves as a substrate for a luminescence-generating technique resulting in the generation of detectable light, allowing the inference that the corresponding target sequence is present.

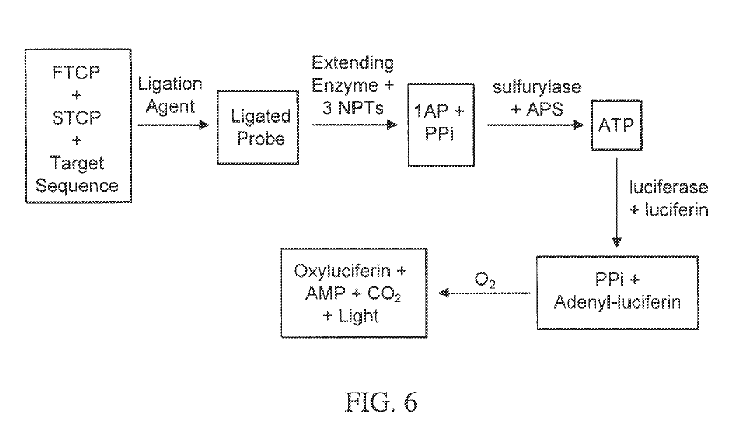

In some embodiments, the free first amplification products 26 (comprising incorporated first primers 24) serve as templates for generating second amplification products (see FIG. 5D). A second primer 27 hybridizes with a free first amplification product 26 and is amplified by a second extending enzyme to generate a second amplification product (shown as a dotted line in FIG. 5D) and PPi, typically in a second reaction composition. The second amplification products can serve as templates for synthesis of additional first amplification products. Such second amplification product and additional first amplification product synthesis can be performed using exponential amplification techniques, including without limitation PCR. In some embodiments, the first extending enzyme is a polymerase comprising strong strand displacement activity, for example but not limited to bacteriophage phi 29 DNA polymerase, Bst DNA polymerase, and T7 DNA polymerase. In some embodiments, the first extending enzyme and the second extending enzyme are the same. In other embodiments, the first extending enzyme and the second extending enzyme are different enzymes. In certain embodiments, the second extending enzyme is a thermostable DNA polymerase, for example but not limited to, Taq DNA polymerase, Deep Vent® DNA polymerase (New England BioLabs, Beverly, Mass.), and Pfu DNA polymerase (Stratagene, La Jolla, Calif.). In some embodiments, the PPi and the additional PPi serve as a substrate for a luminescence-generating technique (see, e.g., FIG. 6).

In certain embodiments, the first and second probes of a probe pair hybridize with the corresponding target sequence and are ligated together, generating a ligated probe. In some embodiments, the ligated probe is released from the ligated probe-target complex. A first primer hybridizes with the corresponding primer-binding site of the ligated probe and selective amplification occurs in a nucleotide-deficient first reaction composition comprising a first extending enzyme to generate a first amplification product which can serve as a template for second amplification product synthesis. In some embodiments, the target sequence is double-stranded and is at least partially denatured to facilitate probe binding. In some embodiments, denaturing a double-stranded sequence includes without limitation, thermal denaturation, chemical denaturation, a helicase, an oligomer comprising a PNA, a stoichiometric excess of primer(s) and/or probe(s), or combinations thereof. In some embodiments, releasing the ligated probe includes without limitation thermal denaturation, chemical denaturation, a helicase, an oligomer comprising a PNA, including without limitation, a first primer comprising a DNA-PNA chimeric oligomer, a stoichiometric excess of primer(s), or combinations thereof.

In certain embodiments, the first amplification products are separated from background sequences; and a second reaction composition is formed comprising the first amplification products, second primers, a second extending enzyme, and all four NTPs. Under appropriate conditions, a second primer hybridizes with a first amplification product and amplification occurs to generate a second amplification product. Those in the art will appreciate that second amplification products can typically serve as templates for synthesis of additional first amplification products. In some embodiments, the second reaction composition further comprises first primers and additional first amplification products and additional second amplification products are generated, typically by exponential amplification, for example but not limited to, PCR.

In some embodiments, the second amplification product, the first and the second amplification products, or surrogates thereof, are detected, indicating that the corresponding target sequence is present in the sample. In some embodiments, the first amplification product, the second amplification product, or the first and the second amplification products comprise a primer binding-site, a reporter probe-binding site, a promoter sequence, a reporter group, a mobility modifier, an affinity tag, a hybridization tag, a ribosome-binding site, or combinations thereof.

Certain methods comprise a step for amplifying a hybridized probe(s) and/or a surrogate of the hybridized probe(s) or for amplifying a ligated probe and/or a surrogate of a ligated probe, including without limitation, a first amplification product and/or a second amplification product; and a step for detecting an amplification product or its surrogate. Some methods comprise a step for selectively amplifying a ligated probe to generate a first amplification product; some methods further comprise a step for amplifying a first amplification product, a second amplification product, surrogates of each, or combinations thereof. Some methods further comprise: a step for denaturing the target sequence or a double-stranded nucleic acid comprising the target sequence, including without limitation a step for generating a strand invasion structure; a step for ligating a probe comprising (i) a first target-complementary portion and a second target-complementary portion, (ii) a probe pair, comprising an first probe and a second probe that each selectively hybridize to the target, (iii) a gap oligonucleotide and either the probe or the probe pair, of (iv) combinations thereof; a step for releasing the ligated probe; a step for generating luminescence; or combinations thereof.

According to certain methods, detection comprises a multiple enzyme luminescence-generating reaction process that is typically performed isothermally, but not always, one embodiment of which is depicted schematically in FIG. 6. First, a ligated probe is generated by a ligation agent, according to the present teachings. The ligated probe is selectively amplified by an extending enzyme to generate a first amplification product (1AP) and inorganic pyrophosphate (PPi) in a first reaction composition. Optionally a second reaction composition is formed and a second amplification product and additional PPi are generated. The PPi is combined with adenosine 5′-phosphosulfate (APS) and a sulfurylase to generate adenosine triphosphate (ATP). Next, the sulfurylase-generated ATP is combined with luciferin and firefly luciferase to form PPi and adenyl-luciferin which is subsequently oxidized to oxyluciferin, generating luminescence. The generated luminescence can be detected using, for example, a luminometer, and the presence of the target sequence in the sample can be inferred through the indirect detection of the amplification product(s).

In certain embodiments of the disclosed methods, a first amplification product is separated from target and background sequences prior to generating a second amplification product. In some embodiments, such separating comprises a pair of affinity tags, a hybridization tag and its complement, or both. In some embodiments, a first amplification product, a second amplification product, or both, comprise: a reporter group, an affinity tag, a primer-binding site, a hybridization tag, a mobility modifier, a reporter probe-binding portion, or combinations thereof.

Ligation according to the present teachings comprises any enzymatic or non-enzymatic means wherein an inter-nucleotide linkage is formed between the opposing ends of a circularizable probe or the opposing ends of the probes of a probe pair that are adjacently hybridized on a target sequence. Typically, the opposing ends of the annealed probe(s) are suitable for ligation (suitability for ligation is a function of the ligation means employed). In certain embodiments, ligation also comprises at least one gap-filling procedure, wherein the ends of the two probes are not adjacently hybridized initially but the 3′-end of the first probe is extended by one or more nucleotide until it is adjacent to the 5′-end of the second probe by an extending enzyme. In other embodiments, the 3′-end of a hybridized circularizable probe is extended until it is adjacent to the 5′-end of the probe in a gap-filling reaction. An internucleotide linkage can then be formed between these adjacent ends by a suitable ligation means. The internucleotide linkage can include, but is not limited to, phosphodiester bond formation. Such bond formation can include, without limitation, those created enzymatically by a DNA ligase, an RNA ligase, or both. Other internucleotide linkages include, without limitation, covalent bond formation between appropriate reactive groups such as between an α-haloacyl group and a phosphothioate group to form a thiophosphorylacetylamino group, a phosphorothioate a tosylate or iodide group to form a 5′-phosphorothioester, and pyrophosphate linkages.

Chemical ligation can, under appropriate conditions, occur spontaneously such as by autoligation. Alternatively, “activating” or reducing agents can be used. Examples of activating and reducing agents include, without limitation, carbodiimide, cyanogen bromide (BrCN), imidazole, 1-methylimidazole/carbodiimide/cystamine, N-cyanoimidazole, dithiothreitol (DTT) and ultraviolet light, such as used for photoligation.

According to the current teachings, ligation generally comprises hybridizing the target-complementary portions of a circularizable probe or of a first probe and a corresponding second probe of a probe pair to the respective complementary regions on the corresponding target sequence; and ligating the 3′ end of the probe (or of the first probe) with the 5′ end of the probe (or of the second probe) to form a ligated probe. The ligated probe is then selectively amplified.

Amplification according to the present teachings encompasses any means by which at least a part of a ligated probe, a first amplification product, a second amplification product, surrogates thereof, or combinations thereof, is reproduced or copied, typically as a complementary strand in a template-dependent manner, including without limitation, a broad range of linear or exponential amplification techniques. Exemplary means for performing an amplifying step include PCR, primer extension, strand displacement amplification (SDA), multiple displacement amplification (MDA), nucleic acid strand-based amplification (NASBA), rolling circle amplification (RCA), transcription-mediated amplification (TMA), single primer isothermal amplification (SPIA™ and Ribo-SPIA™, NuGen Technologies, San Carlos, Calif.), helicase-dependent amplification (HDA), loop-mediated isothermal amplification (LAMP), and the like, including multiplex versions or combinations thereof, for example but not limited to, oligonucleotide ligation assay coupled with RCA (OLA/RCA) and OLA/RCA/PCR. Descriptions of exemplary amplification techniques can be found in, among other places, Sambrook and Russell; Sambrook et al.; Ausbel et al.; PCR Primer: A Laboratory Manual, Diffenbach, Ed., Cold Spring Harbor Press (1995); Rapley; U.S. Pat. No. 6,027,998; PCT Publication Nos. WO 97/31256 and WO 01/92579; Ehrlich et al., Science 252:1643-50 (1991); Vincent et al., EMBO Reports, 5:795-800, 2004; Favis et al., Nature Biotechnology 18:561-64 (2000); and Rabenau et al., Infection 28:97-102 (2000); Lizardi et al., Nat. Genetics 19:225-32, 1998; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40 (1999); and Demidov and Broude. Also within the intended meaning of the term amplification are signal amplification techniques, including without limitation, surrogate amplification methods, branched DNA (bDNA), Hybrid Capture Technology (Digene Corp., Gaithersburg, Md.), Signal Amplification Technology (SAT: Tm Biosciences, Toronto, Canada), and structure-specific nuclease techniques such as Invader technology (Third Wave Technologies, Madison, Wis.). Descriptions of signal amplification techniques can be found in, among other places, Schweitzer and Kingsmore, Curr. Opin. Biotechnol. 12:21-7, 2001; and Andras et al., Mol. Biotechnol. 19:29-44, 2001.

In certain embodiments, amplification comprises a cycle of the sequential steps of: hybridizing a primer with complementary or substantially complementary sequences in a ligated probe, a first amplification product, a second amplification product, or combinations thereof; synthesizing at least one strand of nucleotides in a template-dependent manner using an extending enzyme; and denaturing the newly-formed nucleic acid duplex to separate the strands. The cycle may or may not be repeated. In some embodiments, the synthesizing the strand of nucleotides comprises selective amplification in a nucleotide-deficient reaction composition. Amplification can comprise thermocycling or can be performed isothermally. In certain embodiments, newly-formed nucleic acid duplexes may not be initially denatured, but can be used in their double-stranded form in one or more subsequent steps and either or both strands can, but need not, serve as surrogates of the target sequence. In certain embodiments, single-stranded amplification products are generated and can, but need not, serve as target surrogates.

Primer extension is an amplifying technique that comprises elongating a primer that is annealed to a template, for example a ligated probe or an amplification product, in the 5′=>3′ direction using an amplifying means such as an extending enzyme. According to certain embodiments, under appropriate conditions an extending enzyme can amplify the annealed primer by incorporating nucleotides complementary to the template strand starting at the primer's 3′-end, to generate a complementary strand. In some embodiments, primer extension is carried out in a nucleotide-deficient reaction composition and an amplification product is selectively amplified. In some embodiments, an extending enzyme with strong strand-displacing properties is used as a first extending enzyme for primer extension. In certain embodiments, primer extension can be used to fill a gap between the hybridized ends of a circularizable probe or between two probes of a probe set that are hybridized to a target sequence so that the two probe ends or the probe pair can be ligated together. In certain embodiments, the extending enzyme used for primer extension lacks or substantially lacks 5′-exonuclease activity.

In certain embodiments, an amplification step is performed isothermally. In some embodiments, the isothermal amplifying comprises RCA, including variations of the RCA method. In some embodiments, the amplifying comprises thermocycling, including without limitation, PCR.

Separating comprises any means for removing at least some unreacted components, at least some reagents, or both some unreacted components and some reagents from a ligated probe, a first amplification product, a second amplification product, or combinations thereof. The skilled artisan will appreciate that a number of well-known separation means can be useful in the disclosed methods. Exemplary means/techniques for performing a separation step include gel electrophoresis, for example but not limited to, isoelectric focusing and capillary electrophoresis; dielectrophoresis; flow cytometry, including but not limited to fluorescence-activated sorting techniques using beads, microspheres, or the like; liquid chromatography, including without limitation, HPLC, FPLC, size exclusion (gel filtration) chromatography, affinity chromatography, ion exchange chromatography, hydrophobic interaction chromatography, immunoaffinity chromatography, and reverse phase chromatography; affinity tag binding, such as biotin-avidin, biotin-streptavidin, maltose-maltose binding protein (MBP), and calcium-calcium binding peptide; aptamer-target binding; hybridization tag-hybridization tag complement annealing; mass spectrometry, including without limitation MALDI-TOF, MALDI-TOF-TOF, ESI-TOF, tandem mass spec (MS-MS), LC-MS, and LC-MS/MS; a microfluidic device; and the like. Discussion of separation techniques and separation-detection techniques, can be found in, among other places, Rapley; Sambrook et al.; Sambrook and Russell; Ausbel et al.; Capillary Electrophoresis Theory and Practice, P. Grossman and J. Colburn, eds., Academic Press, 1992; The Expanding Role of Mass Spectrometry in Biotechnology, G. Siuzdak, MCC Press, 2003; PCT Publication No. WO 01/92579; and M. Ladisch, Bioseparations Engineering Principles, Practice, and Economics, John Wiley & Sons, 2001.

In certain embodiments, a separating step comprises a mobility-dependent analytical technique, for example but not limited to capillary electrophoresis. The term “mobility-dependent analytical technique” as used herein, refers to any means for separating different molecular species based on differential rates of migration of those different molecular species in one or more separation techniques. Exemplary mobility-dependent analytical techniques include electrophoresis, chromatography, mass spectrometry, sedimentation, e.g., gradient centrifugation, field-flow fractionation, multi-stage extraction techniques, and the like. Descriptions of mobility-dependent analytical techniques can be found in, among other places, U.S. Pat. Nos. 5,470,705, 5,514,543, 5,580,732, 5,624,800, and 5,807,682; PCT Publication No. WO 01/92579; D. R. Baker, Capillary Electrophoresis, Wiley-Interscience (1995); Biochromatography: Theory and Practice, M. A. Vijayalakshmi, ed., Taylor & Francis, London, U.K. (2003); Krylov and Dovichi, Anal. Chem. 72:111R-128R (2000); Swinney and Bornhop, Electrophoresis 21:1239-50 (2000); Crabtree et al., Electrophoresis 21:1329-35 (2000); and A. Pingoud et al., Biochemical Methods: A Concise Guide for Students and Researchers, Wiley-VCH Verlag GmbH, Weinheim, Germany (2002).

In certain embodiments, a ligated probe, a first amplification product, a second amplification product, or combinations thereof are resolved via a mobility-dependent analytical technique. In certain embodiments, a ligated probe, a first amplification product, a second amplification product, or combinations thereof are resolved (separated) by liquid chromatography. Exemplary stationary phase chromatography media for use in the teachings herein include reversed-phase media (e.g., C-18 or C-8 solid phases), ion-exchange media (particularly anion-exchange media), and hydrophobic interaction media. In certain embodiments, a ligated probe, a first amplification product, a second amplification product, or combinations thereof are separated by micellar electrokinetic capillary chromatography (MECC).

Reversed-phase chromatography is carried out using an isocratic, or more typically, a linear, curved, or stepped solvent gradient, wherein the level of a nonpolar solvent such as acetonitrile or isopropanol in aqueous solvent is increased during a chromatographic run, causing analytes to elute sequentially according to affinity of each analyte for the solid phase. For separating nucleic acid sequences, including ligated probes and amplification products, an ion-pairing agent (e.g., a tetra-alkylammonium) is typically included in the solvent to mask the charge of phosphate.

The mobility of ligated probes, amplification products, and other surrogates can be varied by using mobility modifiers comprising polymer chains that alter the affinity of the probe for the solid, or stationary phase. Thus, with reversed phase chromatography, an increased affinity of the ligated probes and/or amplification products for the stationary phase can be attained by adding a moderately hydrophobic tail (e.g., PEO-containing polymers, short polypeptides, and the like) to the mobility modifier. Longer tails impart greater affinity for the solid phase, and thus require higher non-polar solvent concentration for the amplification products and/or amplification product surrogates to be eluted (and a longer elution time).

In certain embodiments, a ligated probe, a first amplification product, a second amplification product, surrogates thereof, or combinations thereof, are resolved by electrophoresis in a sieving or non-sieving matrix. In certain embodiments, the electrophoretic separation is carried out in a capillary tube by capillary electrophoresis (see, e.g., Capillary Electrophoresis: Theory and Practice, Grossman and Colburn eds., Academic Press, 1992). Exemplary sieving matrices for use in the disclosed teachings include covalently crosslinked matrices, such as polyacrylamide covalently crosslinked with bis-acrylamide; gel matrices formed with linear polymers (see, e.g., U.S. Pat. No. 5,552,028); and gel-free sieving media (see, e.g., U.S. Pat. No. 5,624,800; Hubert and Slater, Electrophoresis, 16: 2137-2142 (1995); Mayer et al., Analytical Chemistry, 66(10): 1777-1780 (1994)). The electrophoresis medium may contain a nucleic acid denaturant, such as 7M formamide, for maintaining polynucleotides in single stranded form. Suitable capillary electrophoresis instrumentation are commercially available, e.g., the ABI PRISM™ Genetic Analyzer series (Applied Biosystems).

In certain embodiments, a hybridization tag complement includes a hybridization enhancer, where, as used herein, the term “hybridization enhancer” means moieties that serve to enhance, stabilize, or otherwise positively influence hybridization between two nucleic acids, e.g. intercalators (see, e.g., U.S. Pat. No. 4,835,263), minor-groove binders (see, e.g., U.S. Pat. No. 5,801,155), and cross-linking functional groups. The hybridization enhancer may be attached to any portion of a mobility modifier, so long as it is attached to the mobility modifier is such a way as to allow interaction with the hybridization tag-hybridization tag complement duplex. In certain embodiments, a hybridization enhancer comprises a minor-groove binder, e.g., netropsin, distamycin, and the like.

The skilled artisan will appreciate that a ligated probe, a first amplification product, a second amplification product, or combinations thereof can also be separated based on molecular weight and length or mobility by, for example, but without limitation, gel electrophoresis, gel filtration, mass spectroscopy, or HPLC, and detected, often in coupled separation-detection techniques. In certain embodiments, a ligated probe, a first amplification product, a second amplification product, or combinations thereof are separated using at least one of the following forces: gravity, electrical, centrifugal, hydraulic, pneumatic, or magnetism.

In certain embodiments, an affinity tag is used to separate the element to which it is bound, e.g., a ligated probe, a first amplification product, a second amplification product, or combinations thereof, from at least one component of the sample, a first reaction composition, a second reaction composition, or combinations thereof. In certain embodiments, an affinity tag is used to bind a ligated probe, a first amplification product, a second amplification product, or combinations thereof to a capture surface, for example but not limited to, a biotinylated ligated probe, a biotinylated amplification product, a biotinylated surrogate, or combinations thereof, to a capture surface comprising streptavidin. In certain embodiments, an aptamer is used to bind a ligated probe, a first amplification product, a second amplification product, a surrogate, or combinations thereof, to a capture surface (see, e.g., Srisawat and Engelke, RNA 7:632-641 (2001); Holeman et al., Fold Des. 3:423-31 (1998); Srisawat et al., Nucl. Acid Res. 29(2):e4, 2001).

In certain embodiments, a hybridization tag, a hybridization tag complement, or a hybridization tag and a hybridization tag complement, is used to separate the element to which it is bound from at least one component of a sample, a reaction composition, or the like. In certain embodiments, hybridization tags are used to attach a ligated probe, a first amplification product, a second amplification product, a surrogate, or combinations thereof, to a capture surface. In certain embodiments, a ligated probe, a first amplification product, a second amplification product, a surrogate, or combinations thereof, comprise the same hybridization tag. For example but not limited to, separating a multiplicity of different element:hybridization tag species using the same hybridization tag complement, tethering a multiplicity of different element:hybridization tag species to a capture surface comprising the same hybridization tag complement, and so forth.

In certain embodiments, a separating step comprises a capture surface, for example but not limited to binding a biotinylated first amplification product, a biotinylated second amplification product, or a biotinylated surrogate of an amplification product to a streptavidin-coated capture surface. In certain embodiments, detecting comprises a capture surface. Suitable capture surfaces include but are not limited to microarrays, appropriately treated or coated reaction vessels and surfaces, beads, for example but not limited to magnetic beads, latex beads, metallic beads, polymer beads, microbeads, and the like (see, e.g., Tong et al., Nat. Biotech. 19:756-59 (2001); Gerry et al., J. Mol. Biol. 292:251-62 (1999); Srisawat et al., Nucl. Acids Res. 29:e4 (2001); Han et al., Nat. Biotech. 19:631-35, 2001; and Stears et al., Nat. Med. 9:140-45, including supplements, 2003). Those in the art will appreciate that the shape and composition of the capture surface is generally not limiting.

In certain embodiments, target surrogates, including without limitation first amplification products and/or second amplification products, are hybridized or attached to a capture surface, including without limitation, a microarray or a bead. In certain embodiments, a capture surface-bound surrogate does not comprise a reporter group, but is indirectly detected due to the hybridization of a labeled entity to the bound surrogate. Such labeled entity include without limitation, a labeled hybridization tag complement, a reporter probe such as a molecular beacon, a light-up probe, a labeled LNA probe, a labeled PNA probe, or a capture probe of the capture surface. In certain embodiments, the labeled entity comprises a fluorescent reporter group and quencher.

The terms “detecting” and “detection” are used in a broad sense herein and encompass any technique by which the presence of the target sequence is determined or inferred. In some embodiments, the presence of a surrogate is detected, directly or indirectly, allowing the presence of the corresponding target sequence to be inferred. For example but not limited to, detecting the luminescence generated by an enzyme cascade reaction (see, e.g., FIG. 6) or detecting the fluorescence generated when a nuclease reporter probe, such as a TaqMan probe is cleaved, wherein the detectable luminescent/fluorescent signal serves as a surrogate for the corresponding amplification product. In some embodiments, such detecting further comprises quantitating the detectable signal, including without limitation, a real-time detection method.

In certain embodiments, a detecting step comprises an instrument, i.e., using an automated or semi-automated detecting means that can, but need not, comprise a computer algorithm. In certain embodiments, a detecting instrument comprises or is coupled to a device for graphically displaying the intensity of an observed or measured parameter of a target surrogate on a graph, monitor, electronic screen, magnetic media, scanner print-out, or other two- or three-dimensional display and/or recording the observed or measured parameter. In certain embodiments, the detecting step is combined with or is a continuation of at least one separating step, for example but not limited to a luminometer coupled with a graphing, recording, or readout component or device; a capillary electrophoresis instrument comprising at least one fluorescent scanner and at least one graphing, recording, or readout component; a chromatography column coupled with an absorbance monitor or fluorescence scanner and a graph recorder; a chromatography column coupled with a mass spectrometer comprising a recording and/or a detection component; or a microarray with a data recording device such as a scanner or CCD camera. In certain embodiments, the detecting step is combined with the amplifying step, for example but not limited to, real-time analysis such as Q-PCR. Exemplary means for performing a detecting step include the ABI PRISM® 3100 Genetic Analyzer, ABI PRISM® 3100-Avant Genetic Analyzer, ABI PRISM® 3700 DNA Analyzer, ABI PRISM® 3730 DNA Analyzer, ABI PRISM® 3730xl DNA Analyzer (all from Applied Biosystems); the ABI PRISM® 7300 Real-Time PCR System; and microarrays and related software such as the ABI PRISM® 1700 (Applied Biosystems) and other commercially available array systems available from Affymetrix, Agilent, and Amersham Biosciences, among others (see also Gerry et al., J. Mol. Biol. 292:251-62, 1999; De Bellis et al., Minerva Biotec 14:247-52, 2002; and Stears et al., Nat. Med. 9:140-45, including supplements, 2003). Exemplary software includes GeneMapper™ Software, GeneScan® Analysis Software, and Genotyper® software (all from Applied Biosystems).

In some embodiments, detecting comprises a handheld device, a manual or visual readout or evaluation, or combinations thereof. In some embodiments, detecting comprises an automated or semi-automated digital or analog readout. In some embodiments, detecting comprises real-time or endpoint analysis. In some embodiments, detecting comprises a microfluidic device, including without limitation, a TaqMan® Low Density Array (Applied Biosystems). In some embodiments, detecting comprises a real-time detection instrument. Exemplary real-time instruments include, the ABI PRISM® 7000 Sequence Detection System, the ABI PRISM® 7700 Sequence Detection System, the Applied Biosystems 7300 Real-Time PCR System, the Applied Biosystems 7500 Real-Time PCR System, the Applied Biosystems 7900 HT Fast Real-Time PCR System (all from Applied Biosystems); the LightCycler™ System (Roche Molecular); the Mx3000P™ Real-Time PCR System, the Mx3005P™ Real-Time PCR System, and the Mx4000® Multiplex Quantitative PCR System (Stratagene, La Jolla, Calif.); and the Smart Cycler System (Cepheid, distributed by Fisher Scientific). Descriptions of real-time instruments can be found in, among other places, their respective manufacturer's users manuals; McPherson; Demidov and Broude; and U.S. Pat. No. 6,814,934.

In certain embodiments, the target surrogates do not comprise fluorescent reporter groups, but are detected and quantified based on their corresponding mass-to-charge ratios (m/z). For example, in some embodiments, a first primer and/or a second primer comprising a mass spectrometry compatible reporter group, including without limitation, mass tags, charge tags, cleavable portions, or isotopes that are incorporated into first amplification products or second amplification products and can be used for mass spectrometer detection (see, e.g., Haff and Smirnov, Nucl. Acids Res. 25:3749-50, 1997; and Sauer et al., Nucl. Acids Res. 31:e63, 2003). The amplification product or a part of the amplification product can be detected by mass spectrometry allowing the presence of the corresponding target sequence to be inferred. In some embodiments, a first primer and/or a second primer, or both comprises a restriction enzyme site, a cleavable portion, or the like, to facilitate release of a part of a subsequent amplification product for detection. In certain embodiments, a multiplicity of surrogates, are separated by liquid chromatography or capillary electrophoresis, subjected to ESI or to MALDI, and detected by mass spectrometry. Descriptions of mass spectrometry can be found in, among other places, The Expanding Role of Mass Spectrometry in Biotechnology, Gary Siuzdak, MCC Press, 2003. Exemplary mass spectrometers for use in the current teachings include the API 2000™ LC/MS/MS System, API 3000™ LC/MS/MS System, API 4000™ LC/MS/MS System, API 4000™ QTRAP™ System, QSTAR® System, QTRAP™ System, Applied Biosystems 4700 Proteomics Analyser, and Voyager™ Biospectrometry™ series instruments (all from Applied Biosystems); Premier and Q-TOF instruments, including associated software and appropriate front-end separation system(s) (Waters); and LTQ series, LCQ series, and Quantum instruments, including associated software and appropriate front-end separation system(s) (ThermoFinnegan).

In certain embodiments, surrogates such as a reporter probe or a cleaved portion of a reporter probe, the reporter group of a released hybridization tag complement, or a part of a hybridization tag complement are detected, directly or indirectly. For example but not limited to, hybridizing a target surrogate to a labeled reporter probe comprising a quencher, including without limitation, a molecular beacon, including stem-loop and stem-free beacons, a TaqMan® probe, a LightSpeed™ PNA probe, or a microarray capture probe. In certain embodiments, the hybridization occurs in solution such as hybridizing a molecular beacon to first amplification product and/or a second amplification product and including surrogates thereof. In other embodiments, a first amplification product, a second amplification product, or a reporter probe is bound to a capture surface and upon hybridization of the corresponding reporter probe, first amplification product, or second amplification product, fluorescence is emitted (see, e.g., EviArrays™ and EviProbes™, Evident Technologies). In certain embodiments, such hybridization events can be simultaneously or near-simultaneously detected.

In certain embodiments, detecting comprises a single-stranded target surrogate, for example but not limited to, detecting a reporter group that is integral to the single-stranded molecule being detected, such as a fluorescent reporter group that is incorporated into a surrogate or the reporter group of a released hybridization tag complement (an exemplary target surrogate); a reporter group on a molecule that hybridizes with the single-stranded target surrogate being detected, such as a hybridization tag complement or reporter probes, including without limitation, PNA beacons, LNA beacons, a TaqMan® probe, a scorpion primer, or a light-up probe.

In certain embodiments, a double-stranded target surrogate is detected, for example but not limited to a first amplification product: second amplification product complex. In some embodiments, such double-stranded surrogates are detected by triplex formation or by local opening of the double-stranded molecule, using for example but without limitation, a PNA opener, a PNA clamp, and triplex forming oligonucleotides (TFOs), either reporter group-labeled or used in conjunction with a labeled entity such as a molecular beacon (see, e.g., Drewe et al., Mol. Cell. Probes 14:269-83, 2000; Zelphati et al., BioTechniques 28:304-15, 2000; Kuhn et al., J. Amer. Chem. Soc. 124:1097-1103, 2002; Knauert and Glazer, Hum. Mol. Genet. 10:2243-2251, 2001; Lohse et al., Bioconj. Chem. 8:503-09, 1997). In certain embodiments, a target surrogate comprises a stretch of homopurine sequences.

In certain embodiments, detecting comprises measuring or quantifying the detectable signal of a reporter group or the change in a detectable signal of a reporter group, typically due to the presence of a target surrogate. For example but not limited to, an unhybridized reporter probe may emit a low level, but detectable signal that quantitatively increases when hybridized, including without limitation, certain molecular beacons, LNA probes, PNA probes, and light-up probes (see, e.g., Svanik et al., Analyt. Biochem. 281:26-35, 2000; Nikiforov and Jeong, Analyt. Biochem. 275:248-53, 1999; and Simeonov and Nikiforov, Nucl. Acids Res. 30:e91, 2002). In certain embodiments, detecting comprises measuring fluorescence polarization. Those in the art understand that the separation means and/or detecting means employed are generally not limiting. Rather, a wide variety of separation means and detecting means are within the scope of the disclosed methods and kits, provided that they allow the presence or absence of the corresponding target sequence(s) to be inferred.

According to the present teachings, a step for generating a ligated probe can be performed using the disclosed ligation agents and/or ligation techniques; a step for selectively amplifying a target sequence, including without limitation a target sequence surrogate, can be performed using a ligated probe, a first primer, a first extending enzyme, and a nucleotide-deficient first reaction composition; a step for generating a first amplification product and a step for generating a second amplification product can be performed using a first primer, a second primer, the disclosed amplifying means, and amplification techniques, including but not limited to selective amplification using a nucleotide-deficient reaction composition; a releasing step can be preformed using the disclosed releasing means, including without limitation, denaturation means; and a step for detecting a target sequence or for detecting a target sequence surrogate can be performed using the disclosed detection means, including instruments.

### IV. CERTAIN KITS

The instant teachings also provide kits designed to expedite performance of the subject methods. Kits serve to expedite the performance of the methods of interest by assembling two or more components required for carrying out the disclosed methods. Kits may contain components in pre-measured unit amounts to minimize the need for measurements by end-users. Kits may include instructions for performing one or more of the disclosed methods. Preferably, the kit components are optimized to operate in conjunction with one another.

In some embodiments, kits comprise a circularizable probe, a probe pair, or both. Some kits comprise a multiplicity of different circularizable probes, a multiplicity of different probe sets, or a multiplicity of different circularizable probes and a multiplicity of different probe sets to selectively amplify and/or detect a multiplicity of different target sequences. Certain kits further comprise a first extending enzyme, a second extending enzyme, a first primer, a second primer, a ligation agent, an ATP sulfurylase, a luciferase, a handheld device for performing certain disclosed methods, a control target sequence, APS, dNTPs, rNTPs, or combinations thereof. In some embodiments, a first extending enzyme, a second extending enzyme, or both, comprises a DNA polymerase, including without limitation, phi29 DNA polymerase, Bst DNA polymerase, a thermostable DNA polymerase, a reverse transcriptase, an RNA polymerase, or combinations thereof. In some embodiments, the ligation means comprises a DNA ligase, an RNA ligase, a chemical ligation means, or a photoligation means. In some embodiments, the luminescence-generating means comprises a luciferase and an appropriate substrate, for example luciferin. In some embodiments, the control target sequence comprises an “internal control” or “standard” sequence for assay calibration and/or validation purposes.

In some embodiments, kits comprise a probe or a probe pair of the current teachings and at least one of: a first primer, a second primer, a ligation means, a first extending means, a second extending means, a releasing means, and a detecting means, including without limitation, a luminescence-generating means.

### V. EXEMPLARY EMBODIMENTS

The current teachings, having been described above, may be better understood by reference to examples. The following exemplary embodiments are intended for illustration purposes only, and should not be construed as limiting the scope of the present teachings in any way.

The current teachings are directed to compositions, methods, and kits for selectively amplifying and for detecting target sequences. In some embodiments, a circularizable probe and/or a probe pair is disclosed for selectively amplifying target sequences. Methods for selectively amplifying target sequences are also disclosed, as are methods for detecting selectively amplified target sequences. Certain embodiments of the disclosed methods comprise a circularizable probe, a probe pair, comprising a first probe and a second probe, a nucleotide-deficient reaction composition, or combinations thereof. In some embodiments, a multiplicity of different circularizable probes, a multiplicity of different probe sets, or a multiplicity of different circularizable probes and a multiplicity of different probe sets are provided to selectively amplify or to detect a multiplicity of different target sequences, typically in a multiplex reaction. According to certain disclosed methods, surrogates of the target sequences are selectively amplified, including without limitation, ligated probes, first amplification products, second amplification products, surrogates thereof, or combinations thereof. In some embodiments, selectively amplified target sequences or their surrogates are detected, directly or indirectly, indicating the presence of the corresponding target sequence.

An exemplary circularizable probe is shown schematically in FIG. 1. The probe 1 comprises a first target-complementary portion 2, a second target-complementary portion 3, and a spacer 4. The probe 1 is shown hybridized with a target sequence 5, in this illustrative embodiment, a segment of listeriolysin gene of Listeria monocytogenes. The nucleotide sequence of the exemplary target sequence is 5′-ACAAATGTGCCGCCAAGAAAAGGTTACAAAGATGGAAATG-3′ (SEQ ID NO:1). The first target-complementary portion 2 and the second target-complementary portion 3 each comprise: (i) the three nucleotides A, C, and T, but not G, and (ii) a universal nucleotide (“N” in FIG. 1). Those in the art will appreciate that a U nucleotide base can typically be incorporated in the probe in place of or in addition to a T nucleotide base without substantially affecting probe hybridization. The spacer 4 comprises ten copies of the hexanucleotide repeat CATTCA (shown as “(CATTCA)₁₀”).

Those in the art will understand that the same universal base may be used throughout the probe or that different universal bases may be incorporated, provided that hybridization of the probe with the target sequence is not substantially destabilized. Those in the art will also understand that spacers can be longer or shorter than 60 nucleotides and need not comprise repeating subunits; and, in this example, any combination of the nucleotide bases (i) C, (ii) A, and (iii) T, U, or T and U can be used, with or without universal bases, however combinations that favor self-hybridization are typically not preferred. It will also be appreciated that the target-complementary portions can be longer or shorter than the illustrative 18-mers depicted, depending at least in part on the target sequence and the hybridization conditions employed.

An exemplary probe pair, comprising a first probe 6 and a second probe 9, hybridized to its corresponding target sequence 5, is depicted in FIG. 2. The first probe 6 comprises a first target-complementary portion 7 and an optional forward primer-binding site 8 (shown as “FPBS”). The second probe 9 comprises a second target-complementary portion 10 and an optional reverse primer-binding site 11 (shown as “RPBS”). The first target-complementary portion 7 and second target-complementary portion 10 of the exemplary probe pair contain (i) a universal base N and (ii) the nucleotides C, G, and T, but not A. Typically the ends of disclosed probes and probe pairs are suitable for ligation or can be made suitable for ligation. For example but not limited to, as shown in FIG. 2, the 3′-end of first probe 6 comprising a hydroxyl group (shown as “3′OH) and the 5′-end of second probe 9 comprising a 5′ phosphate group (shown as “p-5′”) are suitable for ligation when the ligation agent is, for example, a ligase.

In another exemplary embodiment a sample is evaluated for the presence of Listeria monocytogenes. The target sequence, located in the gene encoding the virulence factor “listeriolysin”, is: AAATGTGCCGCCAAGAAAAGGTTACAAAGATGGAAA (SEQ ID NO:2). A synthetic 96-mer circularizable probe with the sequence: TTTCTTNNCNNCACATT(CATTCA)₁₀TTTCCATCTTTNTAACCT (SEQ ID NO:3) is synthesized and 5′-phosphorylated using conventional methodology (see FIG. 1). The first target-complementary portion of the probe is shown underlined, the second target-complementary portion is shown in italics, and the spacer is the 60-mer comprising ten repeats of the hexanucleotide sequence “CATTCA”; “N” is any universal base that allows the probe to hybridize with the target sequence under the conditions employed.

The probe (1 μL of 50 pmol/μL) is combined with 2 μL of sample (25 pmol/μL), 1.475 μL deionized water, 0.5 μL SNPlex ligase buffer (Applied Biosystems), 0.025 μL SNPlex ligase (Applied Biosystems) in a 200 μL microcentrifuge tube, forming a ligation reaction composition. The tube is incubated at 51° C. for two hours in a heating block to generate ligated probe-target sequence complexes, then cooled to 4° C. A first reaction composition, comprising 5 μL phi29 DNA polymerase buffer (New England BioLabs, Beverly Mass.; “NEB”), 0.5 μL of a 10 mg/mL solution of bovine serum albumin (NEB #B9001S), 0.5 μL of a 115 μM solution of APS (Alexis Platforms), 0.5 μL of a 3.2 mU/μL solution of ATP Surfurylase, 5 μL (50 μM) first primer, 5 μL second primer, 21 μL nuclease free water, the ligation reaction composition (5 μL), and 0.75 μL of a 10 U/μL solution of phi29 DNA polymerase (NEB #M0269) is formed in a glass conical vial. The vial is covered with Parafilm and incubated in a water heating block for 10 minutes at 37° C. 2.5 μL of ATP Bioluminescent Assay Mix stock solution (Sigma-Aldrich, Product No. FL-AA) is added to the vial, which is then placed in a TD 20/20 luminometer (Turner BioSystems). A 4 μL volume of alpha-thiol dATP, dGTP, and dTTP (250 pmol each) is aspirated into a Hamilton CR700 constant rate syringe (Hamilton Co., Reno, Nev.) and injected into the luminometer injection port. The remainder of the reaction is performed according to the luminometer instructions and the generated luminescence is detected, indicating that the sample contains L. monocytogenes.

Although the disclosed teachings has been described with reference to various applications, methods, and compositions, it will be appreciated that various changes and modifications may be made without departing from the teachings herein. The foregoing examples are provided to better illustrate the disclosed teachings and are not intended to limit the scope of those teachings.

1-9. (canceled)

10. A method for selectively amplifying a target sequence in the presence of a multiplicity of non-target sequences comprising:
(1) hybridizing a probe with the target sequence, wherein the probe comprises three of the four nucleotide bases: (1) A, (2) C, (3) G, and (4) at least one of T and U, but not the fourth nucleotide base; and
(2) selectively amplifying the target sequence in a first reaction composition comprising:
(a) an extending enzyme and
(b) three of the four nucleotide bases: (1) A, (2) C, (3) G, and (4) at least one of T and U, but not the fourth nucleotide base,
to thereby generate a first amplification product, wherein each of the three nucleotide bases in the reaction composition is the complement of one of the three nucleotide bases in the probe.

11. The method of claim 10, wherein the probe further comprises a universal base.

12. The method of claim 11, wherein the step of selective amplification comprises isothermal amplification.

13. The method of claim 11, wherein the probe further comprises a ligation agent.

14. The method of claim 11, wherein the first amplification product further comprises a moiety selected from the group consisting of a reporter group, an affinity tag, a mobility modifier, a hybridization tag, a primer-binding portion, a reporter probe-binding portion, and combinations thereof.

15. The method of claim 11, further comprising amplifying the first amplification product to generate a second amplification product.

16. The method of claim 15, wherein at least one of the first amplification product and the second amplification product further comprises a moiety selected from the group consisting of a reporter group, an affinity tag, a mobility modifier, a hybridization tag, a primer-binding portion, a reporter probe-binding portion, and combinations thereof.

17. The method of claim 11, further comprising generating a strand invasion structure.

18. The method of claim 17, wherein the strand invasion structure comprises an oligomer comprising PNA.

19. The method of claim 11, wherein the target sequence is double-stranded and is at least partially denatured.

20. The method of claim 19, wherein the denaturing is selected from the group consisting of thermal denaturation, chemical denaturation, a helicase, and combinations thereof.

21. The method of claim 11, further comprising releasing the ligated probe.

22. The method of claim 21, wherein the releasing is selected from the group consisting of thermal denaturation, chemical denaturation, a helicase, a PNA oligomer, a primer comprising a PNA-DNA chimeric oligomer, or combinations thereof.

23. The method of claim 11, wherein the step of selective amplification comprises thermocycling.

24-135. (canceled)
